(19) United States
(12) Patent Application Publication
(10) Pub. No.: US 2009/0162398 A1
(43) Pub. Date: Jun. 25, 2009

(54) Methods and Compositions for Immunizing Pigs Against Porcine Circovirus

(75) Inventor: Stephen Qitu Wu, Fort Dodge, IA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/338,226

(22) Filed: Dec. 18, 2008

Related U.S. Application Data
(60) Provisional application No. 61/015,894, filed on Dec. 21, 2007.

(51) Int. Cl. A61K 39/145; C12N 7/00; C07H 21/00; A61K 35/76; C12N 15/63; C12Q 1/02; A61P 37/04; C12Q 1/68; A61K 35/74; A61K 31/7052; A61K 39/12

(52) U.S. Cl. 424/206.1; 435/235.1; 536/23.72; 424/93.6; 424/93.44; 424/93.4; 514/44; 424/204.1; 435/320.1; 435/6; 435/29

(57) ABSTRACT

The present invention relates to the isolation and identification of two new strains of type 2B porcine circovirus. These two new strains of porcine circovirus may be used for the preparation of vaccine or immunogenic compositions for immunizing pigs against postweaning multisystemic wasting syndrome (PMWS). Accordingly, the invention provides methods for eliciting a protective immune response against a pathogenic porcine circovirus by administering to a pig an immunogenically effective amount of a type 2B porcine circovirus vaccine or immunogenic composition comprising at least one of the porcine circoviruses having a nucleic acid sequence as set forth in SEQ ID NOs: 1 or 2, or at least one protein from at least one of the two new type 2B strains of porcine circovirus as described herein. The invention further relates to protection of a pig from any one or more of the symptoms or sequelae associated with PMWS.

### CROSS-REFERENCE TO RELATED APPLICATIONS

This application claims priority to U.S. provisional patent application No. 61/015,894, filed on Dec. 21, 2007, which is incorporated herein by reference in its entirety.

### FIELD OF THE INVENTION

The present invention relates to the field of animal health and provides methods and compositions for protecting pigs against pathogenic type-2B strains of porcine circovirus. More particularly, the present invention relates to newly identified pathogenic type 2B porcine circovirus strains, the nucleic acid sequences encoding these type 2B strains and the proteins encoded by these nucleic acids. The invention also relates to methods and compositions for eliciting an immune response to a pathogenic porcine circovirus by administering a composition comprising an immunogenically effective amount of at least one of these type 2B porcine circoviruses, or a nucleic acid encoding at least one of these type 2 porcine circoviruses or at least one of the proteins encoded by these nucleic acids.

### BACKGROUND OF THE INVENTION

Porcine circovirus (PCV) is a small icosahedral non-enveloped virus that contains a single stranded circular DNA genome of about 1.76 kb. It was originally isolated as a cell culture contaminant of a porcine kidney cell line PK-15 (I. Tischer et al., Nature 295:64-66 (1982); I. Tischer et al., Zentralbl. Bakteriol. Hyg. Otg. A. 226(2):153-167 (1974)). PCV is classified in the family of Circoviridae, which consists of three other animal circoviruses (chicken anemia virus (CAV), psittacine beak and feather disease virus (PBFDV) and the recently discovered columbid circovirus (CoCV) from pigeons) and three plant circoviruses (banana bunchy top virus, coconut foliar decay virus and subterranean clover stunt virus) (M. R. Bassami et al., Virology 249:453-459 (1998); J. Mankertz et al., Virus Genes 16:267-276 (1998); A. Mankertz et al., Arch. Virol. 145:2469-2479 (2000); B. M. Meehan et al., J. Gen. Virol. 78:221-227 (1997); B. M. Meehan et al., J. Gen. Virol. 79:2171-2179 (1998); D. Todd et al., Arch. Virol. 117:129-135 (1991)). Members of the three previously recognized animal circoviruses (PCV, CAV, and PBFDV) do not share nucleotide sequence homology or antigenic determinants with each other (M. R. Bassami et al., 1998, supra; D. Todd et al., 1991, supra). Experimental infection of pigs with the PK-15 cells-derived PCV did not produce clinical disease and thus, this virus is not considered to be pathogenic to pigs (G. M. Allan et al., Vet. Microbiol. 44:49-64 (1995); I. Tischer et al., Arch. Virol. 91:271-276 (1986)). This nonpathogenic PCV derived from the contaminated PK-15 cell line was designated as porcine circovirus type 1 or PCV1.

Postweaning multisystemic wasting syndrome (PMWS), first described in 1991 (J. C. Harding and E. G. Clark, Swine Health and Production 5:201-203 (1997)), is a complex disease of weaning piglets that is becoming increasingly more widespread. PMWS mainly affects pigs between 5-18 weeks of age. Clinical PMWS signs include progressive weight loss, dyspnea, tachypnea, anemia, diarrhea, and jaundice. Mortality rate may vary from 1% to 2%, and up to 40% in some complicated cases in the U.K. (M. Muirhead, Vet. Rec. 150:456 (2002)). Microscopic lesions characteristic of PMWS include granulomatous interstitial pneumonia, lymphadenopathy, hepatitis, and nephritis (G. M. Allan and J. A. Ellis, J. Vet. Diagn. Invest. 12:3-14 (2000); J. C. Harding and E. G. Clark, Swine Health and Production 5:201-203 (1997)).

While PCV1 is ubiquitous in pigs, it is not pathogenic to pigs. The primary causative agent of PMWS is usually a pathogenic strain of PCV designated as porcine circovirus type 2 or PCV2 (G. M. Allan et al., Vet. Rec. 142:467-468 (1998); G. M. Allan et al., J. Vet. Diagn. Invest. 10:3-10 (1998); G. M. Allan et al., Vet. Microbiol. 66:115-23 (1999); G. M. Allan and J. A. Ellis, J. Vet. Diagn. Invest. 12:3-14 (2000); J. Ellis et al. Can. Vet. J. 39:44-51 (1998); A. L. Hamel et al., J. Virol. 72:5262-5267 (1998); B. M. Meehan et al., 1998, supra; I. Morozov et al., J. Clin. Microbiol. 36:2535-2541 (1998)). The complete genomic sequence of the PMWS-associated PCV2 has been determined (M. Fenaux et al., J. Clin. Microbiol. 38:2494-503 (2000); A. L. Hamel et al., 1998, supra; J. Mankertz et al., 1998, supra; B. M. Meehan et al., 1997, supra; B. M. Meehan et al., 1998, supra; I. Morozov et al., 1998, supra).

Sequence analyses reveals that the PMWS-associated PCV2 shares only about 75% nucleotide sequence identity with the nonpathogenic PCV1. The ORF2 gene of both the nonpathogenic PCV1 and the pathogenic PCV2 encodes for the major immunogenic viral capsid protein (P. Nawagitgul et al., Immunol. Clin. Diagn. Lab Immunol. 1:33-40 (2002); P. Nawagitgul et al., J. Gen. Virol. 81:2281-2287 (2000)).

Due to its potential impact on the pig industry, the development of a vaccine against PCV2 has become of major importance. For example, U.S. Pat. No. 6,287,856 (Poet et al.) and WO 99/45956 describe nucleic acids from psittacine beak and feather disease virus (BFDV), a circovirus that infects avian species, and from porcine circovirus (PCV). The patent proposes vaccine compositions comprising naked DNA or mRNA and discloses a nucleic acid vector for the transient expression of PCV in a eukaryotic cell comprising a cis-acting transcription or translation regulatory sequence derived from the human cytomegalovirus immediate or early gene enhancer or promoter functionally linked to a nucleic acid of the sequence.

U.S. Pat. No. 6,217,883 (Allan et al.) and French Patent No. 2,781,159B describe the isolation of five PCV strains from pulmonary or ganglionic samples taken from pigs infected with PMWS in Canada, California and France (Brittany), and their use in combination with at least one porcine parvovirus antigen in vaccine/immunogenic compositions. While the proteins encoded by PCV2 open reading frames (ORF) consisting of ORF1 to ORF13 are broadly described in the patent, there is no exemplification of any specific protein exhibiting immunogenic properties. The patent further describes vectors consisting of DNA plasmids, linear DNA molecules and recombinant viruses that contain and express in vivo a nucleic acid molecule encoding the PCV antigen.

Several other references, for example, U.S. Pat. No. 6,391,314 B1; U.S. Pat. No. 6,368,601 B1; French Patent No. 2,769,321; French Patent No. 2,769,322; WO 01/96377 A2; WO 00/01409; WO 99/18214; WO 00/77216 A2; WO 01/16330 A2; WO 99/29871; etc., describe the administration of PCV1 or PCV2 polypeptides or the nucleic acids encoding the polypeptides of various strains as vaccine compositions.

The citation of any reference herein should not be deemed as an admission that such reference is available as prior art to the instant invention.

### SUMMARY OF THE INVENTION

In its broadest aspect, the present invention provides the isolation and identification of two new strains of type 2 porcine circoviruses (PCV2), each of which may be used alone, or in combination, for preparation of a vaccine or immunogenic composition for use in protecting pigs against a pathogenic PCV2 infection or for ameliorating at least one symptom associated with Postweaning Multisystemic Wasting Syndrome (PMWS).

Accordingly, a first aspect of the invention provides an isolated porcine type 2 circovirus whose genome comprises the nucleic acid molecule of either of SEQ ID NO: 1 (designated FD07) or 2 (designated FDJE), or whose genome comprises a nucleic acid molecule having at least 95% sequence homology to either of SEQ ID NO: 1 or 2.

In one embodiment, the two newly identified and isolated porcine circoviruses are type 2B porcine circoviruses (PCV2B).

In one embodiment, the isolated porcine circoviruses have an ORF2 protein with at least 92% sequence identity to either of SEQ ID NO: 3 (from FD07) or 4 (from FDJE).

In one embodiment, the isolated porcine circoviruses have an ORF2 protein comprising the amino acid sequence of either of SEQ ID NO: 3 or 4.

A second aspect of the invention provides an isolated nucleic acid molecule encoding a pathogenic type 2B porcine circovirus, or encoding at least one protein from said circovirus, wherein the nucleic acid molecule comprises a nucleotide sequence having at least 95% sequence homology to any of SEQ ID NOs:1 or 2.

In one embodiment, the isolated nucleic acid molecule comprises the nucleotide sequence of any of SEQ ID NOs: 1 or 2.

In one embodiment, an isolated nucleic acid molecule encoding the ORF 1 replicase protein of FD07 comprises residue numbers 51-995 of SEQ ID NO: 5 and an isolated nucleic acid molecule encoding the ORF 2 capsid protein of FD07 comprises residue numbers 1033-1734 of SEQ ID NO: 5.

In one embodiment, an isolated nucleic acid molecule encoding the ORF 1 replicase protein of FDJE comprises residue numbers 51-995 of SEQ ID NO: 6 and an isolated nucleic acid molecule encoding the ORF 2 capsid protein of FDJE comprises residue numbers 1033-1734 of SEQ ID NO: 6.

In one embodiment, the isolated nucleic acid molecule encodes an ORF2 protein having the amino acid sequence as set forth in SEQ ID NO: 3 or 4.

A third aspect of the invention provides an immunogenic or vaccine composition comprising at least one of the following: at least one of the isolated type 2B porcine circoviruses as described herein, or a combination thereof; at least one nucleic acid molecule encoding at least one of the type 2B porcine circoviruses described herein; at least one nucleic acid molecule encoding at least one protein from at least one of the type 2B porcine circoviruses described herein; or at least one protein obtained from at least one of the type 2B porcine circoviruses described herein and a pharmaceutically acceptable adjuvant.

In one embodiment, the vaccine or immunogenic composition can comprise one or more of the following:

a) a live/attenuated, or modified live PCV2B whose genome comprises the nucleic acid molecule of either of SEQ ID NOs: 1 or 2;

b) a killed/inactivated PCV2B whose genome comprises the nucleic acid molecule of either of SEQ ID NOs: 1 or 2;

c) a PCV2B DNA vaccine (e.g. a plasmid vector expressing the ORF2 of PCV2B whose genome comprises the nucleic acid molecule of either of SEQ ID NOs: 1 or 2); or

d) an inactivated viral vector (e.g. a baculovirus, adenovirus, or poxvirus, such as raccoonpox virus; or a bacterium, such as E. coli), that expresses the ORF2 of PCV2B whose genome comprises the nucleic acid molecule of either of SEQ ID NOs:1 or 2.

In one embodiment, a vaccine or immunogenic composition wherein the ORF 2 gene is obtained from a type 2B porcine circovirus of SEQ ID NOs: 1 or 2 may cross-protect against infections with a porcine type 2A, type 2C or type 2D strain, or any other variant. The administering of such vaccine or immunogenic composition results in protecting the pig against low virulence/low mortality type 2A strains, and also results in cross-protection against high virulence/high mortality type 2B strains of pathogenic porcine circoviruses. The vaccine or immunogenic composition utilized may be administered as a single dose or as multiple doses. The administering results in protection of the pig from any one or more of the symptoms or sequelae associated with postweaning multisystemic wasting syndrome (PMWS). Moreover, the administering of the vaccine or immunogenic composition comprising any of the above-noted embodiments also results in reduction in the higher than average mortality associated with the high virulence/high mortality type 2B strains of porcine circovirus.

In one embodiment, the immunogenic or vaccine composition described above may be used for eliciting an immune response against a porcine circovirus, or for protecting pigs against a pathogenic PCV2 infection, or for ameliorating at least one symptom associated with the disease.

In one embodiment, the immunogenic or vaccine composition described above further comprises at least one other microorganism, or an antigen obtained from said microorganism against which an immune response is desired. In one embodiment, the immunogenic or vaccine composition described above further comprises at least one other nucleic acid molecule encoding at least one antigen from at least one other microorganism against which an immune response is desired. The other microorganism may be selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, a pathogen causative of Swine Transmissible Gastroenteritis, and a second different strain of a porcine circovirus. The second different strain of porcine circovirus may be a type 2A or a type 2B circovirus.

In one embodiment, the immunogenic or vaccine composition is administered with or without an adjuvant.

In one embodiment, the immunogenic or vaccine composition is administered in one dose or in multiple doses subcutaneously, intramuscularly, intranasally, transdermally, intrahepatically, or via the intralymphoid route.

A fourth aspect of the invention provides a method of immunizing a pig against viral infection or postweaning multisystemic wasting syndrome (PMWS), or for preventing PMWS in a pig caused by a strain of PCV2, or for ameliorating at least one symptom associated with PMWS, comprising administering to the pig an immunogenically effective amount of a composition comprising any one or more of the following:
a) an immunogenically effective amount of at least one of the type 2 porcine circovirus encoded by the nucleic acid molecule of either of SEQ ID NOs: 1 or 2, as described herein;
b) a nucleic acid molecule encoding at least one of the type 2 porcine circoviruses of a);
c) an immunogenically effective amount of at least one protein isolated from at least one of the type 2 porcine circoviruses of a); or
d) an immunogenically effective amount of at least one nucleic acid molecule encoding at least one protein of c).

In one embodiment, the invention provides methods for immunizing or protecting pigs against at least one pathogenic strain of porcine circovirus by administering a vaccine or immunogenic composition comprising a non-toxic, physiologically acceptable carrier and an immunogenically effective amount of a killed/inactivated type 2 porcine circovirus, or a live, attenuated type 2 porcine circovirus as described herein, whose genome comprises the nucleic acid molecule of either of SEQ ID NOs: 1 or 2. In one embodiment, the methods of the invention provide for immunizing or protecting a pig against a porcine circovirus infection by administering the vaccine or immunogenic composition, as described above, which further comprises an adjuvant.

In one embodiment, the invention provides methods for immunizing or protecting a pig against a pathogenic type 2B strain of porcine circovirus, by administering a vaccine or immunogenic composition comprising an infectious nucleic acid encoding the type 2 porcine circovirus as shown in SEQ ID NO:1 or 2, wherein the administering results in amelioration of one or more symptoms of a porcine circovirus infection.

In one embodiment, the invention provides methods for immunizing or protecting a pig against a pathogenic strain of type 2B porcine circovirus, by administering an immunogenically effective amount of a vaccine or immunogenic composition, wherein the composition comprises at least one protein from the type 2 porcine circovirus as described in the present invention, or a nucleic acid encoding the protein from the type 2 porcine circovirus of the present invention. In one embodiment, the protein from the type 2B porcine circovirus of the present invention is the ORF2 protein. In one embodiment, the ORF-2 gene encoding the ORF2 protein from the type 2B porcine circoviruses of the present invention, designated FD07 and FDJE, comprises residue numbers 1033-1074 of the nucleotide sequence as set forth in SEQ ID NOs: 5 and 6, respectively, and the protein encoded by the ORF-2 gene of FD07 and FDJE comprises the amino acid sequence as set forth in SEQ ID NO: 3 or 4, respectively.

In one embodiment, the invention provides methods for immunizing or protecting a pig against a pathogenic type 2B strain of porcine circovirus, comprising administering a vaccine or immunogenic composition comprising a type 2 porcine circovirus, or a nucleic acid encoding a type 2 porcine circovirus, wherein the porcine circovirus is encoded by the nucleotide sequence as set forth in SEQ ID NO: 1 or 2, their complementary strands, or a nucleic acid sequence having at least 95% homology to the nucleotide sequence of SEQ ID NO: 1 or SEQ ID NO: 2.

In one embodiment, the invention provides methods for immunizing or protecting a pig against a pathogenic type 2B strain of porcine circovirus by administering a vaccine or immunogenic composition comprising at least one of the two new type 2B porcine circoviruses or a nucleic acid encoding at least one of the two new type 2B porcine circoviruses of the present invention, or at least one protein from at least one of the two type 2B strains of the present invention, or the nucleic acid encoding at least one of these two proteins, wherein the pathogenic type 2B strain of porcine circovirus is a strain of porcine circovirus that contains a capsid protein encoded by an ORF 2 gene that exhibits not less than 92% sequence identity with a capsid protein encoded by the ORF 2 gene of at least one of the two strains of the type 2B porcine circoviruses described in the present invention. The amino acid sequences of the capsid proteins of the type 2B strains of porcine circovirus of the present invention are shown in SEQ ID NOs: 3 and 4.

In one embodiment, the methods of the invention provide for immunizing or protecting a pig from infection with a pathogenic strain of type 2B porcine circovirus, comprising administering to a pig a vaccine or immunogenic composition comprising a type 2B porcine circovirus, or a nucleic acid encoding a type 2B porcine circovirus, or encoding at least one protein from said porcine circovirus of the present invention, wherein said administering results in amelioration of one or more of the following clinical symptoms:
reduction of microscopic lesions in one or more lymphoid or non-lymphoid tissues of pigs exposed to a virulent form of a type-2B porcine circovirus;
reduction of viremia associated with a porcine circovirus infection;
reduction in the level of type-2A or type-2B nucleic acid in one or more tissues.

In one embodiment, the method further comprises administering an immunogenically effective amount of a second different immunogenic composition prior to, in conjunction with, or subsequent to, administering the type 2 porcine circovirus immunogenic composition as described herein.

In one embodiment, the second different immunogenic composition comprises an immunogenically effective amount of at least one other microorganism that is pathogenic to pigs, or at least one antigen obtained from said microorganism or a nucleic acid molecule encoding said antigen, wherein the microorganism is selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, a pathogen causative of Swine Transmissible Gastroenteritis and a second different strain of porcine circovirus. The second different strain of porcine circovirus may be a type 2A or a 2B circovirus.

A fifth aspect of the invention provides a vector comprising at least one exogenous nucleic acid molecule encoding a type 2A or a type 2B porcine circovirus protein, wherein the porcine circovirus protein is an ORF2 protein, and wherein the exogenous nucleic acid molecule encoding said protein is set forth in residues 1033-1734 of SEQ ID NO: 5 or 6.

In one embodiment, the vector is a raccoon poxvirus vector containing a nucleic acid molecule encoding at least one protein from a PCV2A, or a PCV2B porcine circovirus as described herein, or both.

In one embodiment, the vector further comprises one or more exogenous nucleic acid molecules encoding an antigen from a microorganism that is pathogenic to pigs, wherein the microorganism is selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, a pathogen causative of Swine Transmissible Gastroenteritis and a second different strain of porcine circovirus.

A sixth aspect of the invention provides a method of determining if a porcine mammal has, or is at risk for developing postweaning multisystemic wasting syndrome (PMWS), the method comprising:

(I) measuring an amount of a PCV2 nucleic acid or protein encoded by said nucleic acid in a tissue sample derived from the mammal, wherein said PCV2 nucleic acid or protein is:
a) a nucleic acid comprising any of SEQ ID NOs: 1, 2, 5 or 6, or a nucleic acid derived therefrom;
b) a protein comprising either of SEQ ID NOs: 3 or 4;
c) a nucleic acid comprising a sequence hybridizable to any of SEQ ID NOs: 1, 2, 5 or 6, or their complements under conditions of high stringency, or a protein comprising a sequence encoded by said hybridizable sequence;
d) a nucleic acid at least 95% homologous to any of SEQ ID NOs: 1, 2, 5, or 6. or their complement as determined using the NBLAST algorithm; or a protein encoded thereby; and
   (II) comparing the amount of said nucleic acid or protein in the tissue sample from the mammal suspected of having, or at risk for developing PMWS with the amount of nucleic acid or protein present in a tissue sample from a normal mammal, or predetermined standard for a normal tissue sample, wherein an elevated amount of said nucleic acid or protein in the tissue sample from the porcine mammal having or suspected of having PMWS compared to the amount in the normal tissue sample or pre-determined standard for a normal tissue sample indicates that the mammal has or is at risk of developing PMWS.

In one embodiment, the method for determining if a porcine mammal has, or is at risk for developing PMWS provides for measuring the amount of the PCV 2B nucleic acid or protein of the invention in a tissue sample selected from the group consisting of inguinal superficial lymph node, tracheobronchial lymph node, submandibular lymph node, lung, tonsil, spleen, liver, kidney, whole blood and blood cells.

### BRIEF DESCRIPTION OF THE DRAWINGS

FIG. 1. Complete genome sequence of type 2B porcine circovirus designated FD07 (SEQ ID NO: 1).

FIG. 2. Complete genome sequence of type 2B porcine circovirus designated FDJE (SEQ ID NO: 2).

FIG. 3. The amino acid sequence of the ORF2 capsid protein of PCV2B designated FD07 (SEQ ID NO: 3).

FIG. 4. The amino acid sequence of the ORF2 capsid protein of PCV2B designated FDJE (SEQ ID NO: 4).

FIG. 5. The nucleic acid sequence encoding the ORF1 and ORF2 proteins of FD07 (SEQ ID NO: 5).

FIG. 6. The nucleic acid sequence encoding the ORF1 and ORF2 proteins of FDJE (SEQ ID NO: 6).

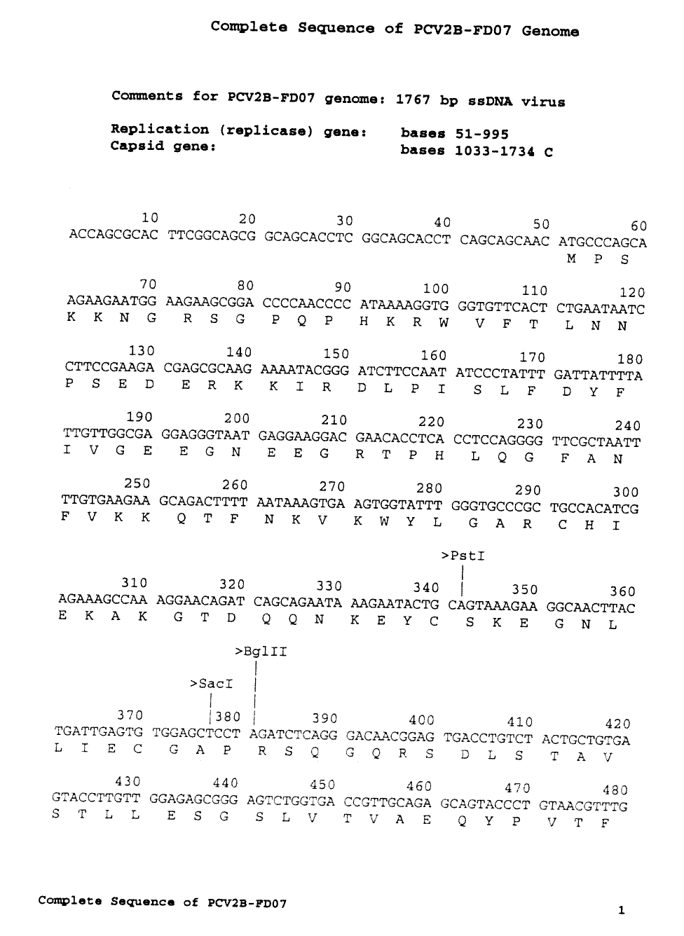

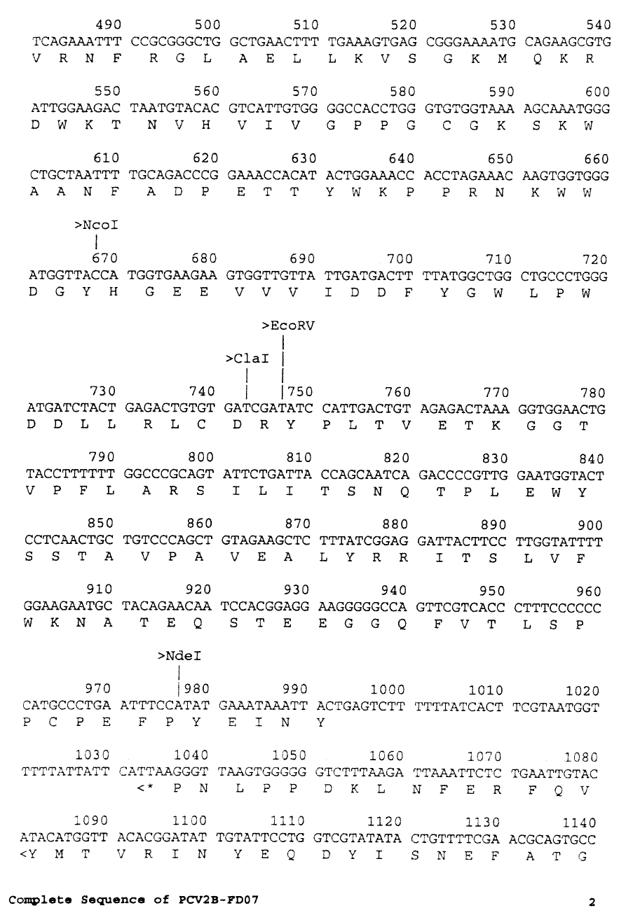

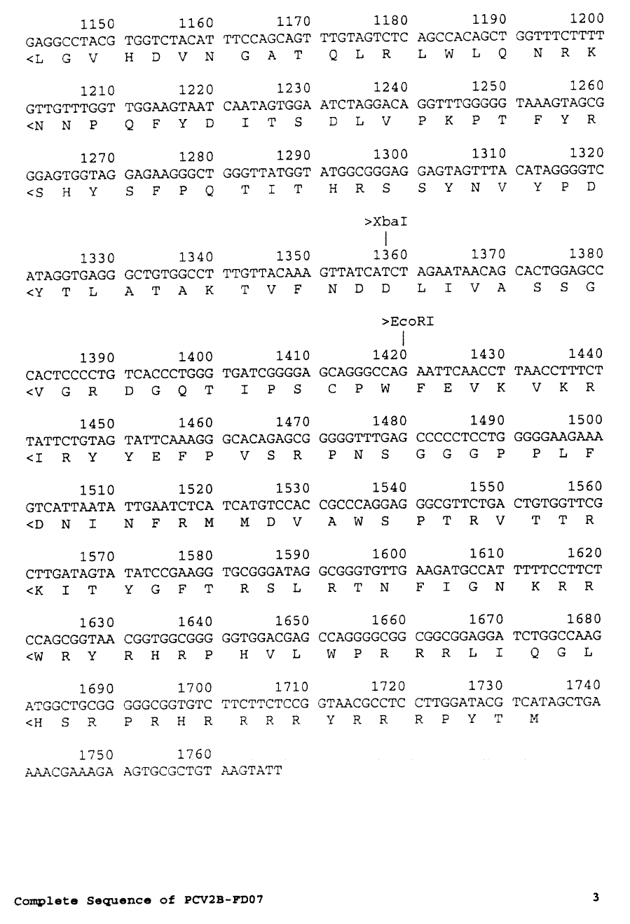

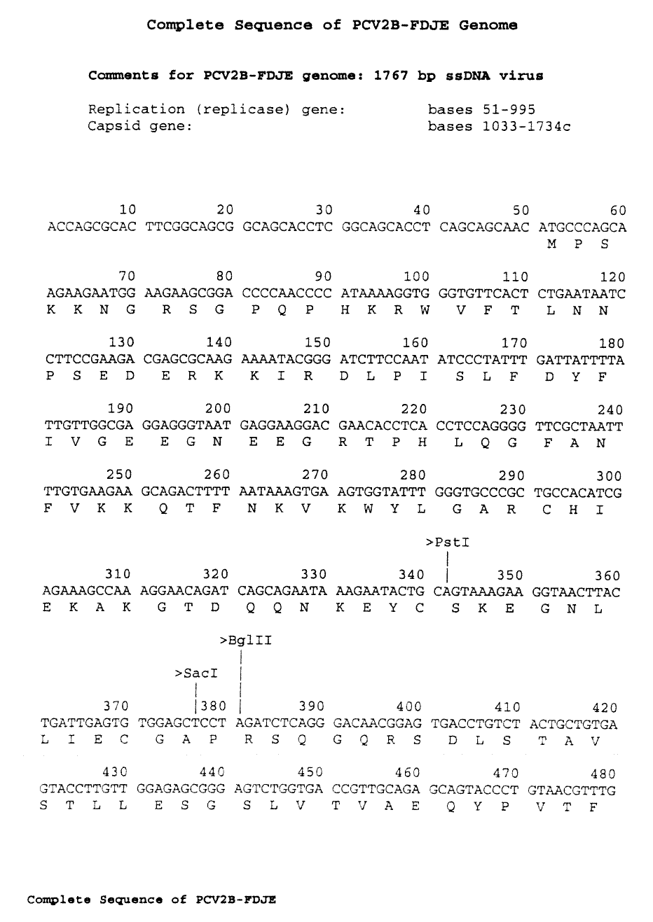

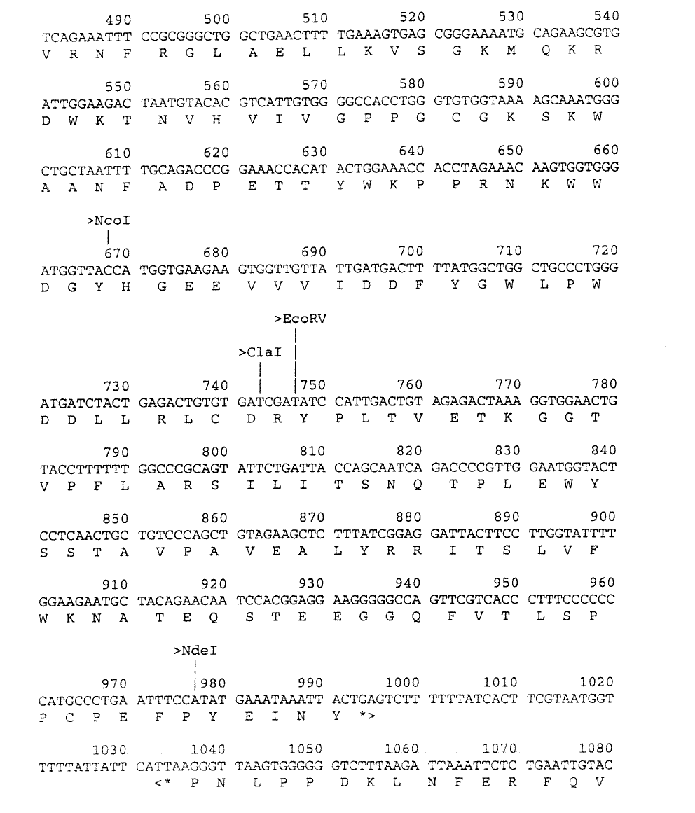

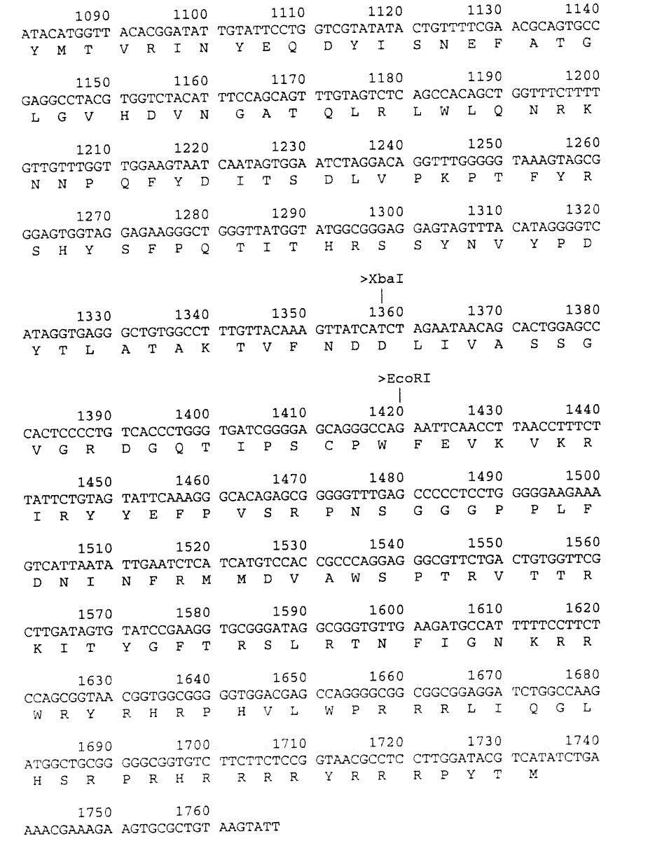

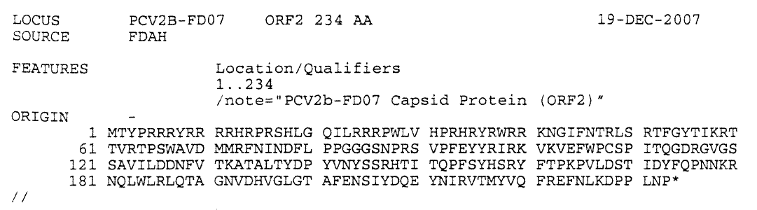

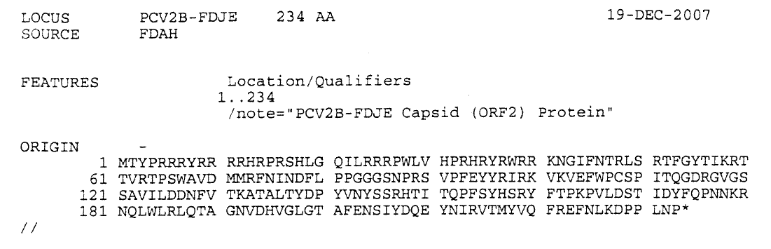

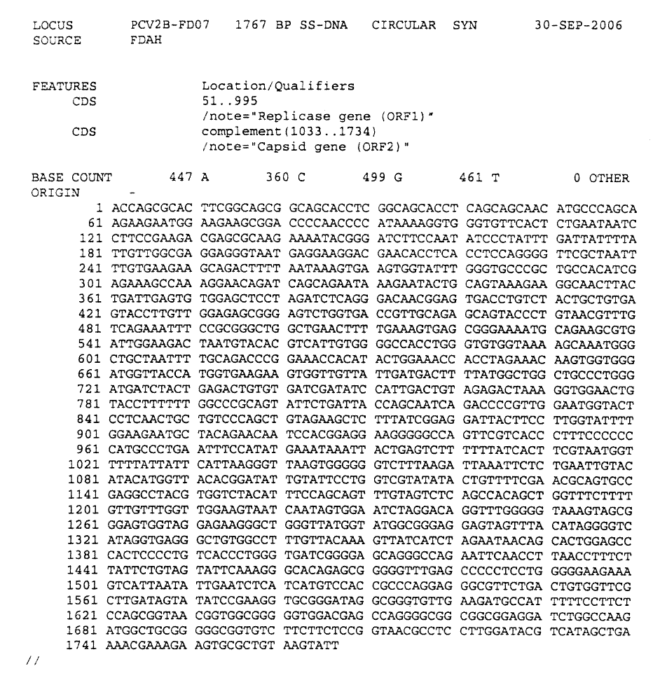

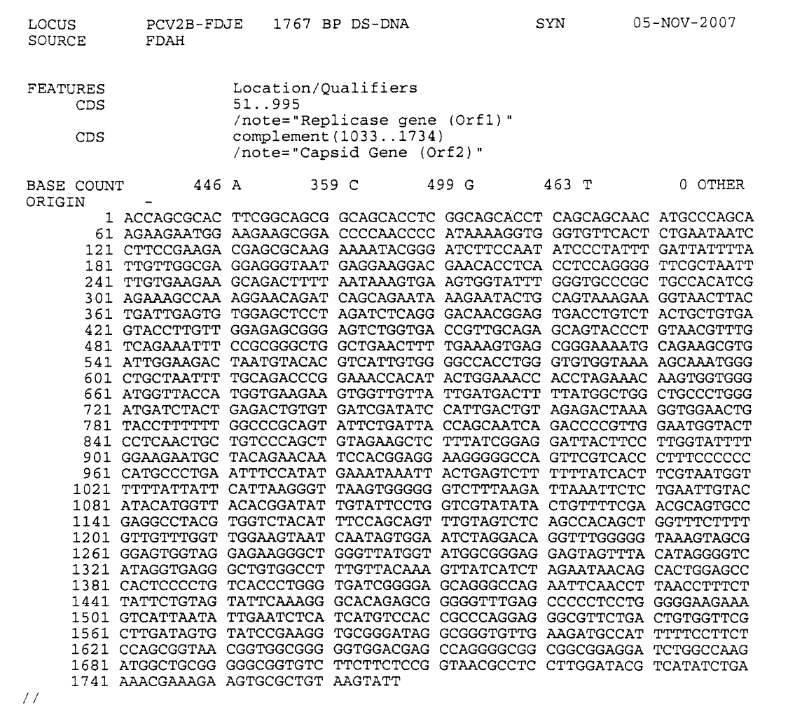

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Accordingly, in the present application, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated by reference in their entirety.

### DEFINITIONS

The terms used herein have the meanings recognized and known to those of skill in the art, however, for convenience and completeness, particular terms and their meanings are set forth below.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Depending on the circumstances, a primary challenge with an antigen alone, in the absence of an adjuvant, may fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

By “antigen” is meant a molecule that contains one or more epitopes capable of stimulating a host's immune system to make a cellular antigen-specific immune response or a humoral antibody response when the antigen is presented in accordance with the present invention. Normally, an epitope will include between about 3-15, generally about 5-15, amino acids. Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Furthermore, for purposes of the present invention, an “antigen” refers to a protein that includes modifications, such as deletions, additions and substitutions (generally conservative in nature, but they may be non-conservative), to the native sequence, so long as the protein maintains the ability to elicit an immunological response. These modifications may be deliberate, as through site-directed mutagenesis, or through particular synthetic procedures, or through a genetic engineering approach, or may be accidental, such as through mutations of hosts, which produce the antigens.

The term “attenuated”, as used herein to describe, for example, an “attenuated virus” and the like refers to a microorganism, for example, a virus, that is limited in its ability to grow or replicate in vitro or in vivo.

The term “circovirus”, as used herein, unless otherwise indicated, refers to any strain of circovirus that falls within the family Circoviridae. For example, in the present invention, the circovirus is a pathogenic porcine circovirus. In particular embodiments, the pathogenic porcine circovirus is a low virulent/low mortality type 2A strain of porcine circovirus or a high virulence/high mortality type 2B strain of porcine circovirus.

“Complementary” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA. Procedures using conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 pg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.).

It is noted that in this disclosure, terms such as “comprises”, “comprised”, “comprising”, “contains”, “containing” and the like can have the meaning attributed to them in U.S. patent law; eg., they can mean “includes”, “included”, “including” and the like. Terms such as “consisting essentially of” and “consists essentially of” have the meaning attributed to them in U.S. patent law, eg., they allow for the inclusion of additional ingredients or steps that do not detract from the novel or basic characteristics of the invention, ie., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, eg., novel, nonobvious, inventive, over the prior art, eg., over documents cited herein or incorporated by reference herein. And, the terms “consists of” and “consisting of” have the meaning ascribed to them in U.S. patent law; namely, that these terms are closed ended.

“Encoded by” or “encoding” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences, which are immunologically identifiable with a polypeptide encoded by the sequence. Thus, an antigen “polypeptide,” “protein,” or “amino acid” sequence may have at least 70% similarity, preferably at least about 80% similarity, more preferably about 90-95% similarity, and most preferably about 99% similarity, to a polypeptide or amino acid sequence of an antigen.

A “gene” as used in the context of the present invention is a sequence of nucleotides in a nucleic acid molecule (chromosome, plasmid, etc.) with which a genetic function is associated. A gene is a hereditary unit, for example of an organism, comprising a polynucleotide sequence (e.g., a DNA sequence for mammals) that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and/or nucleic acids (e.g., phage attachment sites), wherein the gene does not encode an expressed product. Typically, a gene includes coding sequences, such as polypeptide encoding sequences, and non-coding sequences, such as promoter sequences, poly-adenlyation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes).

Thus, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the sequences of the present invention. Therefore, a “homolog” of a porcine circovirus or a fragment thereof, should share at least about 75% homology with the porcine circovirus or fragment thereof (preferably about 80% homology, more preferably about 90-95% homology and most preferably about 99% homology).

An “immune response” to a vaccine or immunogenic composition is the development in a subject of a humoral and/or a cell-mediated immune response to molecules present in the antigen or vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells. A “cell-mediated immune response” is elicited by the presentation of antigenic epitopes in association with Class I or Class II molecules of the major histocompatibility complex (MHC). This activates antigen-specific CD4+ T helper cells or CD8+ cytotoxic T lymphocyte cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to restimulation with antigen. Such assays are well known in the art. See, e.g., Erickson et al., J. Immunol. (1993) 151:4189-4199; Doe et al., Eur. J. Immunol. (1994) 24:2369-2376.

The term “immunogenic” refers to the ability of an antigen or a vaccine to elicit an immune response, either humoral or cell mediated, or both. An “immunogenically effective amount” as used herein refers to the amount of antigen or vaccine sufficient to elicit an immune response, either a cellular (T cell) or humoral (B cell or antibody) response, or both, as measured by standard assays known to one skilled in the art. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum. Furthermore, the level of protection of the immune response may be measured by challenging the immunized host with the antigen that has been injected. For example, if the antigen to which an immune response is desired is a virus or a tumor cell, the level of protection induced by the “immunogenically effective amount” of the antigen is measured by detecting the percent survival or the percent mortality after virus or tumor cell challenge of the animals. In one embodiment, an “immunogenically effective amount” of the vaccine or immunogenic composition refers to a titer of virus particles ranging from about 1 to 7 Log₁₀ virus particles/ml as measured by the FAID₅₀ method (King et al., Journal of Comparative Medicine and Vet. Science, 29:85-89 (1965)) and in U.S. Pat. No. 4,824,785. In one embodiment, an “immunogenically effective amount” of the vaccine or immunogenic compositions is a titer of virus particles ranging from about 2 to 5 Log₁₀ virus particles/ml as measured by the FAID₅₀ method (King et al., Journal of Comparative Medicine and Vet. Science, 29:85-89 (1965)) and in U.S. Pat. No. 4,824,785. In one embodiment, an “immunogenically effective amount” of an infectious DNA vaccine or immunogenic composition may range from about 50 to 5000 μg. In one embodiment, an “immunogenically effective amount” of an infectious DNA vaccine or immunogenic composition may range from about 50 to 1000 μg. In certain embodiments, the term “about” means within 20%, preferably within 10%, and more preferably within 5%.

The term “immunogenic composition” relates to any pharmaceutical composition containing an antigen, eg. a microorganism, which composition can be used to elicit an immune response in a mammal. The immune response can include a T cell response, a B cell response, or both a T cell and B cell response. The composition may serve to sensitize the mammal by the presentation of antigen in association with MHC molecules at the cell surface. In addition, antigen-specific T-lymphocytes or antibodies can be generated to allow for the future protection of an immunized host. An “immunogenic composition” may contain a live, attenuated, or killed/inactivated vaccine comprising a whole microorganism or an immunogenic portion derived therefrom that induces either a cell-mediated (T cell) immune response or an antibody-mediated (B cell) immune response, or both, and may protect the animal from one or more symptoms associated with infection by the microorganism, or may protect the animal from death due to the infection with the microorganism.

An “immunogenic ORF” or “immunogenic ORF” refers to an open reading frame that elicits an immune response, for example, ORF2 encodes an immunogenic capsid protein.

The vaccines and immunogenic compositions of the present invention can further comprise one or more additional “immunomodulators”, which are agents that perturb or alter the immune system, such that either up-regulation or down-regulation of humoral and/or cell-mediated immunity is observed. In one particular embodiment, up-regulation of the humoral and/or cell-mediated arms of the immune system is preferred. Examples of certain immunomodulators include, for example, a “pharmaceutically acceptable adjuvant” or cytokine, among others. Non-limiting examples of “pharmaceutically acceptable adjuvants” that can be used in the vaccine of the present invention include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, and Avridine lipid-amine adjuvant. Non-limiting examples of oil-in-water emulsions useful in the vaccine of the invention include modified SEAM62 and SEAM ½ formulations. Modified SEAM62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM ½ is an oil-in-water emulsion comprising 5% (v/v) squalene, 1% (v/v) SPAN® 85 detergent, 0.7% (v/v) Tween 80 detergent, 2.5% (v/v) ethanol, 100 μg/ml Quil A, and 50 μg/ml cholesterol. Other “immunomodulators” that can be included in the vaccine include, eg., one or more interleukins, interferons, or other known cytokines. In one embodiment, the adjuvant may be a cyclodextrin derivative or a polyanionic polymer, such as those described in U.S. Pat. Nos. 6,165,995 and 6,610,310, respectively.

The term “infectious” means that the virus replicates or is capable of replicating in pigs, regardless of whether or not the virus causes any diseases. In the present invention, an example of an “infectious” DNA is shown as the PCV2 DNA of SEQ ID NOs: 1 or 2.

The term “isolated” or “purified” means that the material is removed from its original environment (e.g., the natural environment if it is naturally occurring). For example, an “isolated” or “purified” peptide or protein is substantially free of cellular material or other contaminating proteins from the cell or tissue source from which the protein is derived, or substantially free of chemical precursors or other chemicals when chemically synthesized. The language “substantially free of cellular material” includes preparations of a polypeptide/protein in which the polypeptide/protein is separated from cellular components of the cells from which it is isolated or recombinantly produced. Thus, a polypeptide/protein that is substantially free of cellular material includes preparations of the polypeptide/protein having less than about 30%, 20%, 10%, 5%, 2.5%, or 1%, (by dry weight) of contaminating protein. When the polypeptide/protein is recombinantly produced, it is also preferably substantially free of culture medium, i.e., culture medium represents less than about 20%, 10%, or 5% of the volume of the protein preparation. When polypeptide/protein is produced by chemical synthesis, it is preferably substantially free of chemical precursors or other chemicals, i.e., it is separated from chemical precursors or other chemicals which are involved in the synthesis of the protein. Accordingly, such preparations of the polypeptide/protein have less than about 30%, 20%, 10%, 5% (by dry weight) of chemical precursors or compounds other than polypeptide/protein fragment of interest. An “isolated” or “purified” nucleic acid molecule is one which is separated from other nucleic acid molecules which are present in the natural source of the nucleic acid molecule. Moreover, an “isolated” nucleic acid molecule, such as a cDNA molecule or an RNA molecule, can be substantially free of other cellular material, or culture medium when produced by recombinant techniques, or substantially free of chemical precursors or other chemicals when chemically synthesized.

The terms “killed” or “inactivated” are used interchangeably herein and refer to a significant or complete reduction in the infectivity of the virus(es) utilized for preparation of the vaccine compositions. The killing or inactivation of the viruses may be evaluated according to any procedure known to those skilled in the art, for example, by molecular biology methods (PCR), methods for titration of the viral titre, fluorescence, immunological methods (ELISA, RIA and the like), immunoenzymatic methods allowing the detection of one or more viral polypeptides (Western and the like). A number of different inactivating agents and means have been employed including formalin, azide, freeze-thaw, sonication, heat treatment, sudden pressure drop, detergent (especially non-ionic detergents), lysozyme, phenol, proteolytic enzymes and .beta.-propiolactone.

The term “lymphoid tissue” refers to any tissue that is rich in lymphocytes and accessory cells such as macrophages and reticular cells and supported by a meshwork of connective tissue. The lymphoid tissue includes the bone marrow, thymus, lymph nodes, spleen, tonsils, adenoids, Peyer's Patches and lymphocyte aggregates on mucosal surfaces. “Non-lymphoid” tissue refers to any other tissue that is not rich in lymphocytes and accessory cells as defined herein.

As used herein, the phrase “nucleic acid” or “nucleic acid molecule” refers to DNA, RNA, as well as any of the known base analogs of DNA and RNA or chimeras formed therefrom. Thus, a “nucleic acid” or a “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5N to 3N direction along the nontranscribed strand of DNA (i.e., the strand having a sequence homologous to the mRNA). A “recombinant DNA molecule” is a DNA molecule that has undergone a molecular biological manipulation.

A “nucleotide” refers to a subunit of DNA or RNA consisting of nitrogenous bases (adenine, guanine, cytosine and thymine), a phosphate molecule, and a sugar molecule (deoxyribose in DNA and ribose in RNA).

The term “open reading frame” or “ORF”, or “ORF”, as used herein, refers to the minimal nucleotide sequence required to encode a particular circovirus protein or antigen without an intervening stop codon.

The term “parenteral” refers to a substance taken into the body or administered in a manner other than through the digestive tract, for example, as by intravenous or intramuscular injection.

The term “pathogenic” refers to the ability of any agent of infection, such as a bacterium or a virus, to cause disease. In the present invention, the term “pathogenic” refers to the ability of a porcine circovirus, in particular, a type 2 porcine circovirus, to cause a disease in pigs referred to as “post-weaning multisystemic wasting syndrome” or “PMWS”. This disease is often characterized by wasting or poor performance in weaned pigs and by moderate to severe lymphoid lesions with lymphoid depletion and histiocytic replacement of follicles in lymphoid tissues. Pigs suffering from PMWS are also known to have respiratory disease, for example, interstitial pneumonia, lymphohistiocytic hepatitis and lymphohistiocytic interstitial nephritis. Other conditions associated with a “pathogenic” type 2 porcine circovirus include sporadic reproductive failure, enteritis, and porcine dermatitis and nephropathy syndrome (PDNS). A “non-pathogenic” microorganism refers to a microorganism that lacks the characteristics noted above for the “pathogenic” strains of porcine circovirus. The “non-pathogenic” porcine circovirus is generally referred to as a type 1 porcine circovirus. The “pathogenic” strains of porcine circovirus are generally referred to as type 2 porcine circoviruses. The “non-pathogenic” porcine circovirus is generally referred to as a type 1 porcine circovirus.

Thus, the term “percent identical” or “percent sequence identity” refers to sequence identity between two amino acid sequences or between two nucleotide sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

The term “pharmaceutically acceptable carrier” means a carrier approved by a regulatory agency of a Federal, a state government, or other regulatory agency, or listed in the U.S. Pharmacopeia or other generally recognized pharmacopeia for use in animals, including humans as well as non-human mammals. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration.

A “polynucleotide” is a nucleic acid polymer, which typically encodes a biologically active (e.g., immunogenic) protein or polypeptide. Depending on the nature of the polypeptide encoded by the polynucleotide, a polynucleotide can include as little as 10 nucleotides, e.g., where the polynucleotide encodes an antigen. Furthermore, a “polynucleotide” can include both double- and single-stranded sequences and refers to, but is not limited to, cDNA from viral, prokaryotic or eukaryotic mRNA, genomic RNA and DNA sequences from viral (e.g. RNA and DNA viruses and retroviruses) or prokaryotic DNA, and also synthetic DNA sequences. The term also captures sequences that include any of the known base analogs of DNA and RNA. The term further includes modifications, such as deletions, additions and substitutions (eg. methylations or capping), to a native sequence, preferably such that the nucleic acid molecule encodes, for example, an antigenic protein. These modifications may be deliberate, as through site-directed mutagenesis, or through particular synthetic procedures, or through a genetic engineering approach, or may be accidental, such as through mutations of hosts, which produce the antigens. The terms “oligonucleotide” or “oligo” are used interchangeably herein.

The terms “porcine” and “swine” are used interchangeably and refer to any animal that is a member of the family Suidae such as, for example, a pig.

The term “protecting” refers to shielding eg. a mammal, in particular, a pig, from infection or a disease, by inducing an immune response to a particular pathogen, eg. circovirus. Such protection is generally achieved following treating a mammal with the vaccine compositions described herein.

The terms “protein”, “polypeptide” and “peptide” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include modifications, such as deletions, additions and substitutions (generally conservative in nature, but which may be non-conservative), to a native sequence, preferably such that the protein maintains the ability to elicit an immunological response within an animal to which the protein is administered. Also included are post-expression modifications, eg. glycosylation, acetylation, phosphorylation and the like.

As used herein, the term “sequence homology” in all its grammatical forms refers to the relationship between proteins that possess a common evolutionary origin, including homologous proteins from different species (Reeck et al., 1987, Cell 50:667).

Two DNA sequences are “substantially homologous” or “substantially similar” when at least about 75% (preferably at least about 80%, and more preferably at least about 90 or 95%, and most preferably about 99%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Similarly, two amino acid sequences are “substantially homologous” or “substantially similar” when greater than 70% of the amino acids are identical, or functionally identical. Preferably, the similar or homologous sequences are identified by alignment using, for example, the GCG (Genetics Computer Group, Program Manual for the GCG Package, Version 7, Madison, Wis.) pileup program.

As used herein, “treatment” (including variations thereof, for example, “treat” or “treated”) refers to any one or more of the following: (i) the prevention of infection or reinfection, as in a traditional vaccine, (ii) the reduction in the severity of, or, in the elimination of symptoms, and (iii) the substantial or complete elimination of the pathogen or disorder in question. Hence, treatment may be effected prophylactically (prior to infection) or therapeutically (following infection). In the present invention, prophylactic treatment is the preferred mode. According to a particular embodiment of the present invention, compositions and methods are provided which treat, including prophylactically and/or therapeutically immunize, a host animal against a viral infection. The methods of the present invention are useful for conferring prophylactic and/or therapeutic immunity to a mammal, preferably a pig. The methods of the present invention can also be practiced on mammals for biomedical research applications.

The terms “vaccine” or “vaccine composition”, which are used interchangeably, refer to pharmaceutical compositions comprising at least one immunogenic composition that induces an immune response in an animal. A vaccine or vaccine composition may protect the animal from disease or possible death due to an infection, and may or may not include one or more additional components that enhance the immunological activity of the active component. A vaccine or vaccine composition may additionally comprise further components typical to pharmaceutical compositions. A vaccine or vaccine composition may additionally comprise further components typical to vaccines or vaccine compositions, including, for example, an adjuvant or an immunomodulator. The immunogenically active component of a vaccine may comprise complete live organisms in either their original form, or as attenuated organisms in a modified live vaccine, or organisms inactivated by appropriate methods in a killed or inactivated vaccine, or subunit vaccines comprising one or more immunogenic components of the virus, or genetically engineered, mutated or cloned vaccines prepared by methods known to those skilled in the art. A vaccine may comprise one or simultaneously more than one of the elements described above. In the present invention, the vaccine compositions include, but are not limited to, live, attenuated or killed/inactivated forms of whole chimeric porcine circoviruses, infectious nucleic acids encoding the chimeric porcine circoviruses, or other infectious DNA vaccines including plasmids, vectors, or other carriers to directly inject DNA into pigs.

### General Description

Due to its potential impact on the pig industry, the development of a vaccine against pathogenic forms of porcine circovirus type 2 (PCV2) is of major importance. It is believed that the nonpathogenic PCV1 will be of limited use against PCV2 infections.

Moreover, new virulent strains of PCV2 have arisen, which are characterized in part by a higher than average mortality rate. These high virulence/high mortality pathogenic strains of PCV2 are designated PCV2B, whereas the low virulence, low mortality pathogenic strains are designated PCV2A. Recently proposed alternate nomenclature for these two strains refers to the PCV2A strain as “Genotype II”, or “RFLP 422”, while the PCV2B strain is referred to as “Genotype I”, or “RFLP 321”. While certain of the previously described vaccine compositions may prove to be effective against the lower mortality, less virulent pathogenic strains of PCV2A, none have been shown to be effective against the high virulence pathogenic PCV2B strains, characterized in part by their higher than average mortality rates.

Given the severity of the infections and the higher than average mortality rate associated with these highly virulent pathogenic PCV2B strains of porcine circovirus, it would be advantageous to identify, isolate and utilize one or more of these strains for the preparation of an immunogenic or vaccine composition for immunization and protection of pigs against postweaning multisystemic wasting syndrome (PMWS).

It is towards the identification and isolation of such strains of PCV2B that the present invention is directed.

In one embodiment, the newly identified and isolated porcine circovirus is a type 2B strain having a nucleic acid sequence as set forth in either of SEQ ID NOs: 1 or 2. In one embodiment, the isolated porcine circovirus is a type 2B strain having a nucleic acid sequence that has at least about 95% sequence homology to that of either SEQ ID NO: 1 or 2. In one embodiment, the ORF2 protein of the newly identified and isolated type 2B porcine circoviruses has at least 92% sequence identity to either of SEQ ID NOs: 3 or 4.

In one embodiment of the present invention, the methods provide for immunizing a pig against a type 2A or 2B pathogenic porcine circovirus (PCV2) comprising administering an immunogenically effective amount of an immunogenic composition comprising a porcine circovirus encoded by the nucleic acids of the present invention.

In particular, the methods of the present invention provide for the use of a vaccine or immunogenic composition comprising one or more of the following:
a) an immunogenically effective amount of at least one of the type 2 porcine circoviruses as described herein, in either attenuated or inactivated/killed form;
b) a nucleic acid molecule encoding at least one of the type 2 porcine circoviruses of a);
c) an immunogenically effective amount of at least one protein isolated from at least one of the type 2 porcine circoviruses of a); or
d) an immunogenically effective amount of at least one nucleic acid molecule encoding at least one protein of c).

In one embodiment, the vaccine or immunogenic composition may comprise a PCV2B DNA vaccine (e.g. a plasmid vector expressing PCV2B ORF2. In one embodiment, the vaccine or immunogenic composition may comprise an inactivated viral vector (e.g. a baculovirus, adenovirus, or poxvirus, such as raccoonpox virus; or a bacterium, such as E. coli), that expresses PCV2B ORF2.

It is also contemplated that the vaccine or immunogenic compositions as described herein are effective for preventing one or more of the symptoms associated with postweaning multisystemic wasting syndrome (PMWS). These symptoms may include, for example, one or more of the following: respiratory disease, microscopic lesions in one or more tissues or organs, histiocytic inflammation, or lymphoid depletion.

Moreover, the vaccines or immunogenic compositions described herein may be used with a second or third vaccine or immunogenic composition that protects pigs against one or more pathogenic porcine viruses or bacteria including: porcine reproductive and respiratory syndrome virus (PRRS), porcine parvovirus (PPV), Mycoplasma hyopneumoniae, Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, and a pathogen causative of Swine Transmissible Gastroenteritis. For example, in one embodiment, the PCV vaccine or immunogenic composition may be combined with a porcine reproductive and respiratory syndrome virus (PRRS) vaccine or immunogenic composition. In one embodiment, the PCV vaccine or immunogenic composition may be combined with a Mycoplasma hyopneumoniae vaccine or immunogenic composition. In one embodiment, the PCV vaccine or immunogenic composition may be combined with a Mycoplasma hyopneumoniae vaccine or immunogenic composition and a porcine reproductive and respiratory syndrome virus (PRRS) vaccine or immunogenic composition.

### Use of the PCV1-2 Vaccines and Immunogenic Compositions

The present invention provides for the identification and isolation of two new pathogenic PCV2B porcine circoviruses. Given the need for a vaccine or immunogenic composition to be administered to pigs to prevent PMWS caused by a pathogenic strain of porcine circovirus, or to ameliorate at least one symptom associated with this disease in pigs, it is envisioned that at least one of these two new strains, or at least one of the nucleic acid encoding these two new strains, or at least one of the proteins obtained from at least one of these strains, may be formulated in a vaccine or immunogenic composition for delivery to a pig in order to immunize the pig against this disease, thus providing protection of pigs against viral infection and postweaning multisystemic wasting syndrome (PMWS).

The vaccine or immunogenic composition comprising at least one of the newly identified and isolated PCV2B porcine circoviruses proposed for use as an immunogenic or vaccine composition in the present studies is prepared using the methods described herein.

### Nucleic Acids of the Invention

The purified and isolated nucleic acid molecules that encode the full-length type 2B pathogenic porcine circovirus, as described herein, are set forth in SEQ ID NOs: 1 and 2. Conventional methods that are well known in the art can be used to make the complementary strands or the nucleotide sequences possessing high homology, for instance, by the art-recognized standard or high stringency hybridization techniques. The purified and isolated nucleic acid molecule comprising the DNA sequence of the immunogenic capsid gene of the PCV2 DNA is set forth in residues 1033-1734 of SEQ ID NOs 5 and 6.

Accordingly, any suitable animal cell containing the PCV2B nucleic acid molecule described herein can produce live, infectious porcine circoviruses. The live, infectious virus is derived from the DNA clone by transfecting, for example, PK-15 cells via in vitro or in vivo. As noted above, one example of the PCV2 DNA is the nucleotide sequence set forth in SEQ ID NOs: 1 and 2. The invention further envisions that the virus may be derived from the complementary strand or a nucleotide sequence having high homology, at least 80%, and more preferably, 95-99% homology, to the nucleotide sequence.

Also included within the scope of the present invention are biologically functional plasmids, viral vectors and the like that contain the nucleic acid molecules described herein, suitable host cells transfected by the vectors comprising the DNA clones and the immunogenic polypeptide expression products. In one embodiment, the immunogenic protein is the capsid protein encoded by ORF2 from a pathogenic type 2B strain of porcine circovirus, as described herein. The amino acid sequence of this capsid protein in the porcine circovirus is set forth in SEQ ID NOs:3 and 4. Biologically active variants thereof are further encompassed by the invention. One of ordinary skill in the art would know how to modify, substitute, delete, etc., amino acid(s) from the polypeptide sequence and produce biologically active variants that retain the same, or substantially the same, activity as the parent sequence without undue effort.

To produce the immunogenic polypeptide products of this invention, the process may include the following steps: growing, under suitable nutrient conditions, prokaryotic or eucaryotic host cells transfected with the nucleic acid molecules described herein in a manner that allows for expression of the polypeptide products, and isolating the desired polypeptide products by standard methods known in the art. It is contemplated that the immunogenic proteins may be prepared by other techniques such as, for example, biochemical synthesis and the like.

### Vaccines and Immunogenic Compositions

The preparation of vaccines or immunogenic compositions comprising the pathogenic PCV2B viruses, as described herein, and methods of using them for protection of pigs against infection with pathogenic strains of porcine circovirus and PMWS, are also included within the scope of the present invention. It is envisioned that the new isolates of PCV2B may be used as a killed/inactivated preparation, or may be attenuated or genetically modified for use to immunize pigs against viral infection and PMWS. Accordingly, the present invention proposes methods to immunize pigs against infection with a pathogenic circovirus or against PMWS using an immunogenically effective amount of a vaccine or immunogenic composition comprising at least one of the two new pathogenic porcine circoviruses described herein, or at least one nucleic acid molecule encoding at least one of these two circoviruses, or at least one protein obtained from at least one of these two circoviruses. The method may protect pigs in need of protection against viral infection or PMWS by administering to the pig an immunogenically effective amount of a vaccine according to the invention, such as, for example, a vaccine comprising an immunogenic amount of the PCV2B DNA, the cloned virus, a plasmid or viral vector containing the DNA of PCV2B, the polypeptide expression products, etc. The vaccine as described herein may be administered with a second or third vaccine or immunogenic composition against other porcine pathogens, including for example, PRRSV, PPV, and other infectious swine agents selected from the following: Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, porcine parvovirus, Escherichia coli, porcine respiratory coronavirus, rotavirus, a pathogen causative of Aujesky's Disease, and a pathogen causative of Swine Transmissible Gastroenteritis antigen. Particular combinations may include a PCV vaccine or immunogenic composition in combination with a PRRSV vaccine or immunogenic composition; a PCV vaccine or immunogenic composition in combination with a Mycoplasma hyopneumoniae vaccine or immunogenic composition; or a PSV vaccine or immunogenic in combination with both of the foregoing vaccines or immunogenic compositions. Immune stimulants may be given concurrently to the pig to provide a broad spectrum of protection against other viral or bacterial infections.

The vaccines or immunogenic compositions used in the methods of the invention are not restricted to any particular type or method of preparation. The vaccines or immunogenic compositions may include, for example, a nucleic acid encoding one or more of the porcine circovirus proteins, infectious DNA vaccines (ie. using plasmids, vectors, or other conventional carriers to directly inject DNA into pigs), live vaccines, modified live vaccines, inactivated vaccines, subunit vaccines, attenuated vaccines, genetically engineered vaccines, etc. In certain embodiments, the vaccine may include the infectious PCV2B DNA, the cloned PCV DNA genome in suitable plasmids or vectors such as, for example, a pSK vector, an avirulent, live virus, an inactivated virus, etc., or a viral vector may be used, such as, but not limited to, a baculovirus vector, an adenovirus vector, or a poxvirus vector, such as raccoonpox virus, or a bacterial vector, such as E. coli. Any of the above may be used in combination with a nontoxic, physiologically acceptable carrier and, optionally, one or more adjuvants.

Inactivated virus vaccines or immunogenic compositions may be prepared by treating the virus derived from the cloned PCV DNA with inactivating agents such as formalin or hydrophobic solvents, acids, etc., by irradiation with ultraviolet light or X-rays, by heating, etc. Inactivation is conducted in a manner understood in the art. For example, in chemical inactivation, a suitable virus sample or serum sample containing the virus is treated for a sufficient length of time with a sufficient amount or concentration of inactivating agent at a sufficiently high (or low, depending on the inactivating agent) temperature or pH to inactivate the virus. Inactivation by heating is conducted at a temperature and for a length of time sufficient to inactivate the virus. Inactivation by irradiation is conducted using a wavelength of light or other energy source for a length of time sufficient to inactivate the virus. The virus is considered inactivated if it is unable to infect a cell susceptible to infection.

The preparation of subunit vaccines typically differs from the preparation of a modified live vaccine or an inactivated vaccine. Prior to preparation of a subunit vaccine, the protective or antigenic components of the vaccine must be identified. Such protective or antigenic components include certain amino acid segments or fragments of the viral capsid proteins which raise a particularly strong protective or immunological response in pigs; single or multiple viral capsid proteins themselves, oligomers thereof, and higher-order associations of the viral capsid proteins which form virus substructures or identifiable parts or units of such substructures; oligoglycosides, glycolipids or glycoproteins present on or near the surface of the virus or in viral substructures such as the lipoproteins or lipid groups associated with the virus, etc. Preferably, a capsid protein, such as the protein encoded by the ORF2 gene, is employed as the antigenic component of the subunit vaccine. Other proteins encoded by the infectious DNA clone may also be used. These immunogenic components are readily identified by methods known in the art. Once identified, the protective or antigenic portions of the virus (i.e., the “subunit”) are subsequently purified and/or cloned by procedures known in the art. The subunit vaccine provides an advantage over other vaccines based on the live virus since the subunit, such as highly purified subunits of the virus, is less toxic than the whole virus.

If the subunit vaccine is produced through recombinant genetic techniques, expression of the cloned subunit such as the ORF2 (capsid) gene, for example, may be optimized by methods known to those in the art (see, for example, Maniatis et al., “Molecular Cloning: A Laboratory Manual,” Cold Spring Harbor Laboratory, Cold Spring Harbor, Mass., 1989). If the subunit being employed represents an intact structural feature of the virus, such as an entire capsid protein, the procedure for its isolation from the virus must then be optimized. In either case, after optimization of the inactivation protocol, the subunit purification protocol may be optimized prior to manufacture.

To prepare attenuated vaccines from pathogenic clones, the tissue culture adapted, live, pathogenic PCV2 is first attenuated (rendered nonpathogenic or harmless) by methods known in the art, typically made by serial passage through cell cultures. Attenuation of pathogenic clones may also be made by gene deletions or viral-producing gene mutations. Then, the attenuated PCV2 viruses may be used to construct additional PCV2 viruses that retain the nonpathogenic phenotype of PCV1 but can vary in the strength of the immunogenicity traits selected from the PCV2 genome through recombinant technology.

Additional genetically engineered vaccines, which are desirable in the present invention, are produced by techniques known in the art. Such techniques involve, but are not limited to, further manipulation of recombinant DNA, modification of or substitutions to the amino acid sequences of the recombinant proteins and the like.

Genetically engineered vaccines based on recombinant DNA technology are made, for instance, by identifying alternative portions of the viral gene encoding proteins responsible for inducing a stronger immune or protective response in pigs (e.g., proteins derived from ORF3, ORF4, etc.). Such identified genes or immuno-dominant fragments can be cloned into standard protein expression vectors, such as the baculovirus vector, and used to infect appropriate host cells (see, for example, O'Reilly et al., “Baculovirus Expression Vectors: A Lab Manual,” Freeman & Co., 1992). The host cells are cultured, thus expressing the desired vaccine proteins, which can be purified to the desired extent and formulated into a suitable vaccine product.

If the clones retain any undesirable natural abilities of causing disease, it is also possible to pinpoint the nucleotide sequences in the viral genome responsible for the virulence, and genetically engineer the virus avirulent through, for example, site-directed mutagenesis. Site-directed mutagenesis is able to add, delete or change one or more nucleotides (see, for instance, Zoller et al., DNA 3:479-488, 1984). An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques (e.g., Sambrook et al., “Molecular Cloning: A Laboratory Manual,” Cold Spring Harbor Laboratory Press, 1989). The cloned virus then exhibits the desired mutation. Alternatively, two oligonucleotides can be synthesized which contain the appropriate mutation. These may be annealed to form double-stranded DNA that can be inserted in the viral DNA to produce full-length DNA.

Genetically engineered proteins, useful in vaccines, for instance, may be expressed in insect cells, yeast cells or mammalian cells. The genetically engineered proteins, which may be purified or isolated by conventional methods, can be directly inoculated into pigs to confer protection against viral infection or postweaning multisystemic wasting syndrome (PMWS) caused by PCV2.

An insect cell line (like HI-FIVE) can be transformed with a transfer vector containing nucleic acid molecules obtained from the virus or copied from the viral genome which encodes one or more of the immuno-dominant proteins of the virus. The transfer vector includes, for example, linearized baculovirus DNA and a plasmid containing the desired polynucleotides. The host cell line may be co-transfected with the linearized baculovirus DNA and a plasmid in order to make a recombinant baculovirus.

Alternatively, DNA from a pig suffering from PMWS, which encode one or more capsid proteins, the infectious PCV2 molecular DNA clone or the cloned PCV DNA genome can be inserted into live vectors, such as a poxvirus or an adenovirus and used as a vaccine.

An immunogenically effective amount of the compositions of the present invention is administered to a pig in need of protection against viral infection or PMWS. The immunogenically effective amount or the immunogenic amount that inoculates the pig can be easily determined or readily titrated by routine testing. An effective amount is one in which a sufficient immunological response to the vaccine is attained to protect the pig exposed to the virus which causes PMWS. Preferably, the pig is protected to an extent in which one to all of the adverse physiological symptoms or effects of the viral disease are significantly reduced, ameliorated or totally prevented.

The vaccine or immunogenic composition can be administered in a single dose or in repeated doses. Dosages may range, for example, from 50 to 5,000 micrograms of the plasmid DNA containing the infectious DNA genome (dependent upon the concentration of the immuno-active component of the vaccine), but should not contain an amount of virus-based antigen sufficient to result in an adverse reaction or physiological symptoms of viral infection. Methods are known in the art for determining or titrating suitable dosages of active antigenic agent based on the weight of the pig, concentration of the antigen and other typical factors. Preferably, the infectious viral DNA clone is used as a vaccine, or a live infectious virus can be generated in vitro and then the live virus may be attenuated and then used as a vaccine. In that case, 100 to 200 micrograms of cloned PCV DNA or about 10,000 50% tissue culture infective dose (TCID₅₀) of live attenuated virus can be given to a pig.

Desirably, the vaccine or immunogenic composition is administered to a pig not yet exposed to the PCV virus. The vaccine containing the PCV2 infectious DNA clone or other antigenic forms thereof can conveniently be administered intranasally, transdermally (i.e., applied on or at the skin surface for systemic absorption), parenterally, etc. The parenteral route of administration includes, but is not limited to, intramuscular, intravenous, intraperitoneal, intradermal (i.e., injected or otherwise placed under the skin) routes and the like. Since the intramuscular and intradermal routes of inoculation have been successful in other studies using viral infectious DNA clones (E. E. Sparger et al., “Infection of cats by injection with DNA of feline immunodeficiency virus molecular clone,” Virology 238:157-160 (1997); L. Willems et al., “In vivo transfection of bovine leukemia provirus into sheep,” Virology 189:775-777 (1992)), these routes are most preferred, in addition to the practical intranasal route of administration. Although less convenient, it is also contemplated that the vaccine is given to the pig through the intralymphoid route of inoculation. A unique, highly preferred method of administration involves directly injecting the plasmid DNA containing PCV2 or the PCV2 virus (attenuated or inactivated) into the pig intramuscularly, intradermally, intralymphoidly, etc.

When administered as a liquid, the present vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable “physiologically acceptable” carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of porcine body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

In one embodiment, the immunogenic ORF2 capsid gene derived from at least one of the type 2B circoviruses described herein may be used in the vaccine or immunogenic composition.

### Adjuvants

The present invention provides for the isolation and identification of two new type 2B porcine circoviruses (PCV2B) encoded by the nucleic acid sequences of SEQ ID NOs: 1 and 2. These new strains of PCV2B were isolated from pigs who were environmentally exposed and as such, may be ideal candidates for use in preparation of vaccine and immunogenic compositions. It is an object of the invention to utilize these strains in killed/inactivated forms, or to prepare attenuated forms of these two strains for preparation of vaccine or immunogenic compositions. It is also an object of the invention to deliver them with or without an adjuvant to a population of pigs to prevent PMWS or to ameliorate at least one symptom associated with this disease.

Accordingly, a live, attenuated type 2B porcine circovirus, or a killed/inactivated porcine circoviruses, as described herein, or the nucleic acid encoding these porcine circoviruses, or at least one protein obtained from these circoviruses may be delivered with or without an adjuvant. In one embodiment, the vaccine is a killed/inactivated PCV2B circovirus, which is administered with an adjuvant. An adjuvant is a substance that increases the immunological response of the pig to the vaccine. The adjuvant may be administered at the same time and at the same site as the vaccine, or at a different time, for example, as a booster. Adjuvants also may advantageously be administered to the pig in a manner or at a site different from the manner or site in which the vaccine is administered. Suitable adjuvants include, but are not limited to, aluminum hydroxide (alum), immunostimulating complexes (ISCOMS), non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

### Assays for Measuring Immune Responses

The functional outcome of vaccinating a pig against porcine circovirus can be assessed by suitable assays that monitor induction of cellular or humoral immunity or T cell activity. These assays are known to one skilled in the art, but may include measurement of cytolytic T cell activity using for example, a chromium release assay. Alternatively, T cell proliferative assays may be used as an indication of immune reactivity or lack thereof. In addition, in vivo studies can be done to assess the level of protection in a mammal vaccinated against a pathogen using the methods of the present invention. Typical in vivo assays may involve vaccinating an animal with an antigen, such as the chimeric porcine circovirus described herein. After waiting for a time sufficient for induction of an antibody or T cell response to occur, generally from about one to two weeks after injection, the animals will be challenged with the antigen, such as either a virus, and amelioration of one or more symptoms associated with the viral infection, or survival of the animals is monitored. A successful vaccination regimen against porcine circovirus will result in significant decrease in one or more symptoms associated with the viral infection, or a decrease in viremia, or a decrease in the number or severity of lesions associated with a viral infection, or survival when compared to the non-vaccinated controls. Serum may also be collected to monitor levels of antibodies generated in response to the vaccine injections, as measured by methods known to those skilled in the art.

### Methods for Comparing Porcine Circovirus Type 2A and Type 2β Isolates

It may be possible that the newly identified type 2B porcine circoviruses may be effective as vaccine or immunogenic compositions to protect against infection with either a type 2A or a type 2B circovirus, both of which are pathogenic in pigs. Type 2A and type 2B strains may be differentiated through use of restriction fragment length polymorphism (RFLP) analysis. RFLP uses enzyme digestion of viral nucleic acid (partial or whole), which results in a specific cutting pattern that is visualized on a gel. If there are differences between viruses at the site of enzyme cutting, different patterns can be observed. This fingerprinting technique has been commonly used for DNA viruses. Meng et al. (U.S. patent publication 2005/0147966) describe the use of a PCR-RFLP assay using the NcoI restriction enzyme to distinguish between non-pathogenic type 1 porcine circoviruses and pathogenic type 2 porcine circoviruses. An ORF2 based PCR-RFLP assay described in 2000 using HinfI, HinP1I, KpnI, MseI, and RsaI enzymes is able to distinguish among PCV2 isolates (PCV2A, B, C, D, and E) (Hamel A L, Lin L L, Sachvie C, Grudeski E, Nayar G P: PCR detection and characterization of type-2 porcine circovirus. Can J Vet Res. 64:44-52, 2000).

An ORF2 based PCR-RFLP assay using Sau3AI, BanII, NspI, XbaI, and CfrI enzymes has been described recently and is able to distinguish 9 different PCV2 genotypes (Wen L, Guo X, Yang H: Genotyping of porcine circovirus type 2 from a variety of clinical conditions in China. Vet Microbiol. 110:141-146, 2005). PCV2 RFLP analysis showed that there was a significant change from RFLP type 422 to type 321 in 2005 in Ontario, Canada (Delay J, McEwen B, Carman S, van Dreuel T, Fairles J: Porcine circovirus type 2-associated disease is increasing. AHL Newsletter. 9:22, 2005).

In addition to using RFLP analysis to differentiate between type 2A and type 2B porcine circoviruses, it is believed that these two strains may be differentiated on the basis of sequences analysis.

For example, with sequence analysis it is possible to characterize the genetic information and compare isolates to each other (Choi J, Stevenson G W, Kiupel M, Harrach B, Anothayanontha L, Kanitz C L, Mittal S K: Sequence analysis of old and new strains of porcine circovirus associated with congenital tremors in pigs and their comparison with strains involved with postweaning multisystemic wasting syndrome. Can J Vet Res. 66:217-224, 2002; De Boisséson C, Béven V, Bigarré L, Thiéry R, Rose N, Eveno E, Madec F, Jestin A: Molecular characterization of porcine circovirus type 2 isolates from post-weaning multisystemic wasting syndrome-affected and non-affected pigs. J Gen Virol. 85:293-304, 2004; Fenaux M, Halbur P G, Gill M, Toth T E, Meng X J: Genetic characterization of type 2 porcine circovirus (PCV-2) from pigs with postweaning multisystemic wasting syndrome in different geographic regions of North America and development of a differential PCR-restriction fragment length polymorphism assay to detect and differentiate between infections with PCV-1 and PCV-2. J Clin Microbiol. 38:2494-2503, 2000; Grierson S S, King D P, Sandvik T, Hicks D, Spencer Y, Drew T W, Banks M: Detection and genetic typing of type 2 porcine circovirus in archived pig tissues from the UK. Arch Virol. 149:1171-1183, 2004; Kim J H, Lyoo Y S: Genetic characterization of porcine circovirus-2 field isolates from PMWS pigs. J Vet Sci. 3:31-39, 2002; Mankertz A, Domingo M, Folch J M, LeCann P, Jestin A, Segales J, Chmielewicz B, Plana-Durán J, Soike D: Characterisation of PCV-2 isolates from Spain, Germany and France. Virus Res. 66:65-77, 2000). To further investigate possible differences among PCV2 isolates it is possible to sequence the entire PCV2 genome or to sequence only ORF2.

The two strains also differ with respect to the pathology, clinical symptoms and mortality associated with the disease itself, with type 2A demonstrating less severe lesions in bodily tissues and a lower mortality rate, as compared to the more severe lesions and higher mortality rate associated with type 2B strains. These clinical parameters may be measured using standard procedures known in the art and as demonstrated in the present invention.

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. All parts and percents referred to herein are on a weight basis and all temperatures are expressed in degrees centigrade unless otherwise specified.

### Example 1

### Isolation and Identification of Two New Type 2B Strains of Porcine Circovirus

A study was planned to test a new vaccine formulation in pigs to assess its efficacy against porcine circovirus and M. hyopneumoniae. During the course of the study, it was observed that several of the pigs in the control and vaccinated groups exhibited symptoms of PMWS. It was then confirmed that these pigs were exposed to environmental PCV2 prior to challenge. Molecular analysis on blood and tissue samples from these pigs revealed that they harbored a type 2B strain that was different than the strain used for challenge. Moreover, sequence analysis established that the PCV2B strain designated FD07 and another new PCV2B strain isolated from a pig on the farm (designated FDJE) were different than other type 2B strains identified in other field studies and from those previously identified by others. The materials and methods for the isolation and characterization of these two new strains of PCV2B are described below.

### Materials and Methods

### Test Animals

Mixed breed male and female conventional pigs were used for this study. There were 24 vaccinates and 24 controls. The pigs were 3 weeks of age at the time of the vaccination.

The pigs for the study were purchased from commercial farm and identified using an ear tag. Pigs were obtained from a single source herd. At the time of the first vaccination pigs were seronegative to PCV2 as determined by ELISA (S/P ratio≦0.5). The target population for the study was healthy feeder pigs. With respect to their immune function, the animals selected for this study were deemed as representative of feeder pigs in the United States.

The pigs were housed in isolation facilities at FDAH, Fort Dodge, Iowa. Vaccinates and controls were intermingled throughout the whole study in similar environments. The piglets were blocked into rooms by litter. Housing space was in compliance with applicable regulations of animal welfare. The pigs were fed a standard commercial diet with water and food available ad libitum.

Pigs requiring medical attention were treated as deemed necessary by the plant veterinarian after consultation with the study investigator.

PCV2-PCR testing results of serum samples indicated accidental/environmental PCV2 exposure to piglets in one of the rooms prior to the experimental challenge. The PCV2 was first detected in the serum of one control piglet at 28 days post vaccination, and identified as a porcine circovirus type 2B strain (PCV2B) by DNA sequencing. This PCV2B infected an additional 6 piglets. Two of the unvaccinated control pigs developed PCV2-associated clinical signs and were euthanized at 18 days post challenge due to poor health conditions. The clinical signs included, but were not limited to, inappetence, lethargy, depression, sneezing, coughing, nasal discharge, ocular discharge and dyspnea. Prior to being euthanized, tissue and blood samples were collected for circovirus analysis and sequencing.

### Sample Collection and Testing

### Sample Collection

### Nasal Swabs

Nasal swabs were collected on 0 days post vaccination (DPV) for PCV2 isolation to ensure there was no PCV2 infection in the tested animals prior to vaccination. The nasal swab samples were placed into individual sterile tubes containing 3 mL of MEM with lactalbumin hydrolysate (LAH) and gentamicin (60 μg/mL), penicillin (100 U/mL) and streptomycin (100 μg/mL), and stored at or below −50° C. until tested.

### Serum Samples

Pigs were bled for serum samples (no more than ten mL) at 0, 13, 28 DPV, and −1, 7, 14, 20 DPC for ELISA testing, and at 0, 13, 28 DPV, −1, 3, 7, 10, 14, 17, 20 DPC for PCR testing.

### Tissue Samples

All animals were necropsied at 20/21 DPC. Sections from three lymph nodes (tracheobronchial, iliac and inguinal), tonsil, and spleen were collected and fixed in formalin for histopathology examination and PCV2 immunohistochemistry (IHC) testing.

### Sample Testing

### Enzyme-Linked Immunosorbent Assay (ELISA)

Indirect ELISA was used to detect anti-PCV2 antibodies in serum samples using recombinant PCV2 capsid protein (expressed in baculovirus) as capture antigen. Briefly, a 96-well polystyrene plate was coated with positive capture antigen (Sf9 cells infected with recombinant baculovirus expressing PCV2 capsid protein). Six wells on each plate were coated with negative capture antigen (SF9 insect cells) as a control. After treating the plate with blocking reagent the plate was incubated with test and control serum samples. Each sample of test serum was added into the wells of an immunoplate coated with Positive Capture Antigen (3 wells per each test sample). Positive control serum sample was added into the six wells of immunoplate coated with Positive Capture Antigen (positive control) and six wells of immunoplate coated with Negative Capture Antigen (negative control). Then the HRP conjugated goat anti-swine secondary antibody was added to the plate. Finally, TMB (peroxidase substrate) was added and incubated for an appropriate and consistent time. The color developed was quantified with an ELISA plate reader. Each reagent in the ELISA plate was incubated in a well-defined manner and was washed to remove excess reagent prior to each successive step. The OD of test samples and positive control was calculated by subtracting average OD of negative control from average OD of test samples and positive control. Serum titer was expressed as S/P (sample/positive control) ratio, that is, OD of the test sample divided by that of the positive control sample.

### PCR Testing

PCV2-specific PCR testing was used to detect the presence of PCV2 viral genomic DNA in serum samples. Viral genomic DNA was purified from serum using QIAGEN MatAttract Virus Mini Kit and QIAGEN BioRobot M48 Workstation. To detect PCV2 specific sequences, a 592-bp fragment was amplified by using ABI AmpliTaq Gold DNA polymerase and gene-specific primers: F1PCV2, 5′-ATGCCCAGCAAGAA GAATGG-3′ (SEQ ID NO:7) and RPCV2,5′-TGGTTTCCAGTATGT GGTTTCC-3′ (SEQ ID NO: 8). The purified viral DNA was used as template and denatured at 95° C. for 10 min. The PCR program of reactions consisted of 35 cycles of denaturation at 94° C. for 30 sec, annealing at 59° C. for 1 min, and extension at 72° C. for 1 min, Ten μL of PCR product were used to detect 592 bp PCV2 DNA fragment by agarose gel electrophoresis.

### Histopathology

Tissue samples of spleen, tonsil, and three (tracheobronchial, iliac and inguinal) lymph nodes, were collected from each animal during necropsy, fixed in 10% neutral buffered formalin for 2 to 4 days and embedded in paraffin. Four-micrometer sections were stained with hematoxylin and eosin and examined under the light microscope for histopathologic evaluation.

The samples were evaluated for degree of lymphocyte depletion/histiocytic replacement. Briefly, all tissues were examined in a blinded fashion and given subjective scores for the level of lymphocyte depletion and histiocytic replacement. Ranked score of lymphoid depletion from 0 to 3 was assigned as follows: 0—normal, 1—mild lymphoid depletion with loss of overall cellularity, 2—moderate lymphoid depletion, 3—severe lymphoid depletion with loss of lymphoid follicle structure. Ranked score of histiocytic replacement from 0 to 3 was assigned in a similar way as follows: 0—normal, 1—mild histiocytic-to-granulomatous inflammation, 2—moderate histiocytic-to-granulomatous inflammation, 3—severe histiocytic-to-granulomatous inflammation with replacement of follicles.

Histopathological evaluations were conducted by a certified pathologist at the Veterinary Diagnostic Laboratory, College of Veterinary Medicine, Iowa State University (Ames, Iowa).

### Immunohistochemistry (IHC) Testing

PCV2-specific antigen in lymph nodes, tonsil and spleen tissues was detected by immunohistochemistry testing. Briefly, four-micrometer sections of tissues were placed on glass slides and treated with xylene to remove paraffin. Deparaffinized sections were quenched for endogenous peroxidase with 3% H₂O₂ in PBS for 20 min. After rinsing with distilled water the sections were incubated at room temperature overnight with rabbit anti PCV2 polyclonal serum diluted 1:1,000 in PBS. After washing with PBS the sections then were incubated with biothynilated goat anti-rabbit IgG for 30 min at room temperature. Then sections were incubated with streptavidin peroxidase conjugate and “visualized” with diaminobenzidine tetrahydrochloride substrate. The amount of PCV2 antigen was assessed in a blinded fashion based on the level of PCV2 antigen staining. The ranked score was assigned as follows: 0—no staining, 1—low level of staining, 2—medium level of staining, and 3—high level of staining. The IHC on tissues was conducted at the Veterinary Diagnostic Laboratory in the Iowa State University College of Veterinary Medicine (Ames, Iowa).

### Results

### Virus Isolation

Nasal swabs were collected on 0 days post vaccination (DPV) for PCV2 isolation to ensure there was no PCV2 infection in the test animals prior to vaccination. No virus was isolated from the nasal swabs of each test animal, indicating the lack of exposure of these pigs to environmental PCV2 infection at the time of the vaccination.

### PCV2 Challenge Material Titration

The titration results of the virulent PCV2 challenge material (strain 40895) are shown in Table 1. The average titer of challenge virus from triplicate titrations was 4.6 Log₁₀ FAID₅₀/mL.

### Serology

The results of PCV2-specific antibody ELISA are shown in Table 2.

On 0 DPV1, ELISA S/P ratios in controls (Group C) varied from −0.009 to 0.366 with an average S/P ratio of 0.135. Following vaccination, the ELISA antibody titers in the controls declined to background levels by 13 DPV and all piglets remained seronegative until the day of challenge. The average S/P ratios were 0.049 on 13 DPV, 0.023 on 28 DPV, and 0.004 on 35 DPV (−1 DPC), respectively. Following PCV2 challenge the average S/P ratios significantly increased from 0.125 on 7 DPC, 0.612 on 14 DPC, to 0.922 on 20 DPC.

On 0 DPV1, ELISA S/P ratios in vaccinates (Group V) varied from −0.028 to 0.364 with average S/P ratio of 0.126. Following vaccination the average S/P ratios were 0.041 on 13 DPV, 0.097 on 28 DPV, and 0.167 on 35 DPV (−1 DPC). Following PCV2 challenge, a significant boost in ELISA titers in piglets of this group was observed as early as by 7 DPC, with average S/P ratios of 1.069 on 7 DPC, 1.373 on 14 DPC, and 1.356 on 20 DPC.

### PCV2 Viremia

The detection of PCV2 viremia by PCV2-specific PCR in sera of vaccinates and controls is summarized in Table 3A. This PCV2-specific PCR is at least 1,000 times more sensitive than the conventional cell culture method.

On −1 days post challenge (DPC), unexpectedly, it was found that 3 out of 24 vaccinates (Pigs #P102, P103, and P104) and 6 out of 24 controls (Pigs # G197, G205, 0162, P107, P108 and P110) were detected as positive for PCV2 DNA in the serum samples while all others remained negative. All 9 PCV2 positive pigs were housed in the same room (#12). Unfortunately six PCV2-infected pigs (Pig #P102, P103, P104, P107, P108 and P110) and two non-infected pigs were relocated into Room #13 and comingled with 8 non-infected pigs from Room #11 at −1 DPC due to space requirements. Therefore, all animals in Rooms #12 and 13 were potentially exposed to environmental PCV2.

To investigate the source of PCV2 environmental exposure, the serum samples collected on 0, 13, and 28 DPV, were tested by PCV2-specific PCR. All pigs were PCV2 negative except the Pig #P108 (Control, Room #12) with an extremely strong positive PCR band on 13 DPV. This result suggested that accidental environmental PCV2 exposure originated from Pig #P108, and then spread to the other pigs in Room #12 and #13. The exact origin of this PCV2 is not known, however, most likely from the environment or the farm in which Pig #P108 was infected at undetectable level prior to enrolling in the study.

To determine the genotype of the PCV2 from the environmental exposure, the PCR products from Pig #P108 were cloned for sequencing. DNA sequence analysis indicated that PCV2 from Pig #P108 was a type 2B strain, different from strain-#40895 (2A). The genomes of these two PCV2 strains shared 95.98% DNA sequence identity. To further differentiate experimental PCV2 (strain #40895) challenge from environmental contact challenge (2B from Pig #P108), PCR products from all PCV2 viremic pigs were cloned for DNA sequencing. The results of the genotyping are shown in the Column “PCV2 Challenge”: “A” represents that no PCV2B was detected in the serum samples and the challenge took place only by experimental PCV2-#40895 strain; “A+B” represents that in addition to experimental challenge, PCV2B was also detected and all PCV2B were from Pig #P108 due to identical sequence; and “A+(B)” represents that in addition to experimental challenge, pigs were potentially infected with PCV2B because these pigs were comingled with Pig #P108 in Room No. 12/13. However, all of these pigs were protected from PCV2 challenge.

In order to evaluate the impact of the experimental and environmental contact challenges, the PCR results for PCV2 viremia are discussed in three categories: overall comparison (Table 3A), experimental challenge (Table 3B), and experimental and environmental contact challenges (Table 3C).

Overall comparison between groups showed that 10/24 (41.7%) vaccinates were positive for the presence of PCV2 DNA in sera, for at least a single positive and inconclusive occurrences. If a single inconclusive occurrence is not accountable, 5/24 (20.8%) vaccinates were PCV2 DNA positive. In contrast, 24/24 (100%) controls were positive for the presence of PCV2 DNA in sera for multiple occurrences. The frequency and density of the PCV2 positive band in the controls were significantly higher and stronger than in the vaccinates (see Table 3A).

Comparison between groups exposed to PCV2 experimental challenge (Table 3B) showed that 1/9 (11.1%) vaccinates were PCV2 DNA positive for only a single positive occurrence. In contrast, 14/14 (100%) controls were positive for the presence of PCV2 DNA in sera for multiple occurrences.

Comparison between groups exposed to PCV2 experimental and environmental contact challenges (Table 3C) showed that 4/15 (26.7%) vaccinates were PCV2 DNA positive, for at least a single positive occurrence. In contrast, 10/10 (100%) controls were positive for the presence of PCV2 DNA in sera for multiple occurrences.

### Microscopic Lesions-Lymphoid Depletion

Tissues of lymph nodes, spleen and tonsil were examined microscopically for lymphoid depletion. The total results of microscopic lesions-lymphoid depletion are summarized in Table 4A (number of animals with score 0-3).

Overall comparison between groups showed that an abnormal lymphoid depletion score in at least one tissue was observed in 15/24 (62.5%) of vaccinated pigs, compared to 23/24 (95.8%) of control pigs. Moderate to severe lymphoid depletion lesions were observed in 4/24 (16.7%) vaccinates, compared to 14/24 (58.3%) controls.

Comparison between groups exposed to PCV2 experimental challenge (Table 4B) showed that an abnormal lymphoid depletion score in at least one tissue was observed in 6/9 (66.7%) of vaccinated pigs, compared to 13/14 (92.9%) of control pigs.

Comparison between groups exposed to PCV2 experimental and environmental contact challenges (Table 4C) showed that an abnormal lymphoid depletion score in at least one tissue was observed in 9/15 (60%) of vaccinated pigs, compared to 10/10 (100%) of control pigs.

### Microscopic Lesions-Histiocytic Replacement

Tissues of lymph nodes, spleen and tonsil were examined microscopically for histiocytic-to-granulomatous inflammation with replacement of follicles. The total results of microscopic lesions-histiocytic replacement are summarized in Table 5.

Overall comparison showed that compared to 22/24 (91.7%) of controls, 15/24 (62.5%) vaccinates were observed with abnormal histiocytic replacement score in at least one lymph tissue.

### Immunohistochemistry

The results of detection of amount of PCV2 antigen by IHC staining in lymph nodes, spleen and tonsil are summarized in Table 6 (number of animals with score 0-3).

Overall comparison showed that compared to 23/24 (95.8%) of controls, 7/24 (29.2%) vaccinates were the PCV2 specific IHC staining in at least one lymph tissue.

### Post-Challenge Clinical Observations

The results of the daily observations for post-challenge clinical signs in individual pigs are presented in Table 7. No clinical signs were observed in any pig except 2 control pigs (#P108 and O162).

Pig #P108 was observed with coughing on 1 DPC, diarrhea on 10 DPC, thin, diarrhea and inactive on 17 DPC. This pig developed obvious clinical signs of wasting. Due to the poor condition the piglet was euthanized on 18 DPC. Necropsy revealed very little subcutaneous fat, moderate cranioventral consolidation in lungs, empty stomach and intestines. This pig developed PCV-associated disease, supported by the findings in PCV2 viremia (extremely strong PCR positive), severe lymphoid depletion, and high load of PCV2 antigen (by IHC staining) in lymph tissues.

Piglet #O162 was observed to be thin and lame in the left hind leg on 14 DPC. On 17 DPC this pig was observed again to be thin, unable to move, and with swollen tarsal joints in the hind legs. Due to the poor condition the piglet was euthanized on 18 DPC. Necropsy revealed moderate to severe lung consolidation with fibrinous adhesions. Microscopic evaluation of lung tissues showed severe acute bronchopneumonia and chronic focal interstitial fibrosis. This pig developed PCV-associated disease, supported by the findings in PCV2 viremia (extremely strong PCR positive), severe lymphoid depletion, and high load of PCV2 antigen (by IHC staining) in lymph tissues.

TABLE 1
PCV2 (#40895) Challenge Material Titration

| Replicate | *Titer |
|---|---|
| 1 | 4.6 |
| 2 | 4.8 |
| 3 | 4.4 |
| 4 | 4.6 |
| 5 | 4.6 |
| Ave ± SD | 4.6 ± 0.14 |

*Log₁₀ FAID₅₀ per mL
Ave ± SD = Average titer ± Standard Deviation

TABLE 2
Seroconversion to PCV2 in Sera of Vaccinated and Control
Pigs Measured by PCV2 Antibody ELISA*

| PIG ID | GROUP | 0 DPV | 13 DPV | 28 DPV | −1 DPC | 7 DPC | 14 DPC | 20 DPC |
|---|---|---|---|---|---|---|---|---|
| G199 | V | 0.029 | 0.022 | 0.425 | 0.883 | 1.298 | 1.367 | 1.358 |
| G203 | V | 0.010 | −0.015 | −0.007 | 0.004 | 0.591 | 1.373 | 1.458 |
| G204 | V | 0.038 | −0.002 | 0.057 | 0.034 | 1.399 | 1.524 | 1.524 |
| G289 | V | 0.121 | 0.055 | 0.021 | 0.034 | 0.938 | 1.530 | 1.536 |
| G290 | V | 0.162 | 0.035 | 0.041 | 0.027 | 1.007 | 1.438 | 1.451 |
| O112 | V | 0.222 | 0.062 | 0.059 | 0.017 | 0.520 | 1.145 | 1.127 |
| O113 | V | 0.050 | 0.007 | 0.057 | 0.112 | 1.164 | 1.460 | 1.395 |
| O117 | V | −0.028 | −0.010 | 0.381 | 0.608 | 1.264 | 1.334 | 1.282 |
| O119 | V | 0.030 | −0.007 | 0.001 | −0.013 | 0.935 | 1.209 | 1.196 |
| O156 | V | 0.058 | 0.008 | 0.158 | 0.272 | 1.434 | 1.438 | 1.416 |
| O158 | V | 0.099 | 0.039 | 0.023 | 0.128 | 1.451 | 1.462 | 1.447 |
| O159 | V | 0.082 | 0.034 | 0.184 | 0.234 | 1.194 | 1.433 | 1.415 |
| P102 | V | 0.108 | 0.028 | 0.026 | 0.017 | 1.199 | 1.307 | 1.321 |
| P103 | V | 0.206 | 0.096 | 0.177 | 0.238 | 1.322 | 1.457 | 1.371 |
| P104 | V | 0.139 | 0.046 | 0.023 | 0.034 | 1.351 | 1.438 | 1.394 |
| P109 | V | 0.179 | 0.048 | 0.041 | 0.630 | 1.396 | 1.492 | 1.409 |
| P113 | V | 0.113 | 0.026 | 0.039 | 0.011 | 0.980 | 1.251 | 1.368 |
| P115 | V | 0.141 | 0.077 | 0.055 | 0.077 | 1.266 | 1.436 | 1.353 |
| P145 | V | 0.069 | −0.003 | 0.120 | 0.219 | 1.215 | 1.521 | 1.500 |
| P146 | V | 0.158 | 0.065 | 0.030 | 0.037 | 0.213 | 1.372 | 1.273 |
| P150 | V | 0.124 | 0.049 | 0.009 | 0.108 | 0.619 | 1.265 | 1.306 |
| P192 | V | 0.288 | 0.113 | 0.173 | 0.072 | 0.697 | 1.399 | 1.314 |
| P213 | V | 0.364 | 0.121 | 0.116 | 0.085 | 0.909 | 0.847 | 0.967 |
| P214 | V | 0.264 | 0.097 | 0.110 | 0.135 | 1.299 | 1.444 | 1.373 |
| Ave (S/PRatio) | | 0.126 | 0.041 | 0.097 | 0.167 | 1.069 | 1.373 | 1.356 |
| | | 0.094 | 0.039 | 0.111 | 0.228 | 0.334 | 0.150 | 0.127 |
| G197 | C | 0.072 | 0.005 | 0.017 | −0.005 | 1.011 | 1.135 | 1.240 |
| G205 | C | 0.028 | −0.010 | −0.003 | −0.016 | 0.389 | 1.072 | 1.042 |
| G293 | C | 0.202 | 0.033 | 0.019 | −0.011 | −0.017 | 0.376 | 0.949 |
| G296 | C | 0.266 | 0.052 | 0.032 | −0.001 | 0.016 | 0.932 | 1.073 |
| O109 | C | 0.014 | −0.007 | −0.012 | −0.013 | 0.009 | 0.701 | 1.254 |
| O110 | C | 0.001 | −0.015 | −0.017 | −0.003 | −0.032 | 0.355 | 0.704 |
| O111 | C | −0.002 | −0.019 | −0.021 | −0.033 | −0.034 | 0.448 | 0.858 |
| O114 | C | −0.009 | −0.014 | −0.019 | −0.028 | −0.041 | 0.159 | 0.216 |
| O162 | C | 0.219 | 0.040 | 0.039 | 0.032 | 0.216 | 0.536 | NA |
| O164 | C | 0.057 | 0.018 | −0.005 | −0.007 | 0.019 | 0.227 | 0.675 |
| O166 | C | 0.072 | 0.003 | −0.003 | −0.009 | 0.041 | 0.914 | 1.146 |
| P105 | C | 0.228 | 0.234 | 0.035 | 0.022 | 0.134 | 1.376 | 1.428 |
| P107 | C | 0.142 | 0.043 | 0.037 | 0.001 | 0.549 | 1.102 | 1.195 |
| P108 | C | 0.266 | 0.080 | 0.039 | −0.029 | −0.036 | −0.029 | NA |
| P110 | C | 0.204 | 0.047 | 0.027 | 0.024 | 0.574 | 0.704 | 0.772 |
| P112 | C | 0.069 | 0.132 | 0.036 | 0.019 | 0.023 | 0.783 | 1.170 |
| P116 | C | 0.080 | 0.023 | 0.006 | 0.012 | −0.005 | 0.338 | 0.841 |
| P147 | C | 0.149 | 0.024 | 0.029 | −0.006 | −0.005 | 0.199 | 0.597 |
| P148 | C | 0.033 | 0.008 | 0.016 | −0.016 | 0.006 | 0.642 | 0.966 |
| P194 | C | 0.343 | 0.136 | 0.106 | 0.042 | 0.070 | 0.208 | 0.633 |
| P195 | C | 0.169 | 0.100 | 0.046 | 0.021 | 0.028 | 0.816 | 1.093 |
| P215 | C | 0.110 | 0.037 | 0.032 | 0.013 | 0.029 | 0.433 | 0.826 |
| P216 | C | 0.171 | 0.071 | 0.044 | 0.035 | 0.036 | 0.600 | 0.906 |
| P218 | C | 0.366 | 0.147 | 0.082 | 0.043 | 0.027 | 0.664 | 0.701 |

| | | | | | | | |
|---|---|---|---|---|---|---|---|
| Ave (S/P Ratio) | 0.135 | 0.049 | 0.023 | 0.004 | 0.125 | 0.612 | 0.922 |
| | 0.109 | 0.063 | 0.031 | 0.022 | 0.256 | 0.358 | 0.277 |

*Results expressed as sample/positive control (S/P) ratios; Ave (S/P Ratio) ± SD = Average S/P Ratio ± Standard Deviation
NA = not determined;
V = Vaccinate Group;
C = Control Group

TABLE 3A
Detection of PCV2 Viremia by PCV2-Specific PCR in Sera of Vaccinated and Control Pigs: Overall Comparison

| PIG | | | PCV2 | 0 | 13 | 28 | −1 | 3 | 7 | 10 | 14 | 17 | 20 | PCV2 |
|---|---|---|---|---|---|---|---|---|---|---|---|---|---|---|
| ID | GROUP | ROOM | CHALLENGE | DPV | DPV | DPV | DPC | DPC | DPC | DPC | DPC | DPC | DPC | Viremia |
| G199 | V | 12 | A + (B) | − | − | − | − | − | − | − | − | − | − | − |
| G203 | V | 12 | A + B | − | − | − | − | + | ++ | ++++ | +++ | ++ | ++ | P |
| G204 | V | 12 | A + B | − | − | − | − | ++ | ++ | ++ | ++ | + | + | P |
| G289 | V | 11 | A | − | − | − | − | − | ± | − | − | − | − | P* |
| G290 | V | 11 | A | − | − | − | − | − | ± | − | − | − | − | P* |
| O112 | V | 11-13 | A + (B) | − | − | − | − | − | − | − | − | − | − | − |
| O113 | V | 11-13 | A + (B) | − | − | − | − | − | − | − | − | − | − | − |
| O117 | V | 11-13 | A + (B) | − | − | − | − | − | − | − | − | − | − | − |
| O119 | V | 11-13 | A | − | − | − | − | − | − | + | − | − | − | P |
| O156 | V | 12 | A + (B) | − | − | − | − | − | − | − | − | − | − | − |
| O158 | V | 12 | A + (B) | − | − | − | − | − | − | − | − | − | − | − |
| O159 | V | 12 | A + (B) | − | − | − | − | − | − | − | − | − | − | − |
| P102 | V | 12-13 | A + B | − | − | − | + | − | − | − | − | − | − | P |
| P103 | V | 12-13 | A + B | − | − | − | ± | − | − | − | − | − | − | P* |
| P104 | V | 12-13 | A + B | − | − | − | ++ | ++++ | ++ | +++ | +++ | + | ± | P |
| P109 | V | 12-13 | A + (B) | − | − | − | − | − | − | − | − | − | − | − |
| P113 | V | 11 | A | − | − | − | − | − | − | − | − | − | − | − |
| P115 | V | 11 | A | − | − | − | − | − | − | − | − | − | − | − |
| P145 | V | 11 | A | − | − | − | − | − | − | − | − | − | − | − |
| P146 | V | 11 | A | − | − | − | − | − | ± | − | − | − | ± | P* |
| P150 | V | 11 | A | − | − | − | − | − | − | − | − | − | − | − |
| P192 | V | 11 | A | − | − | − | − | − | − | − | − | − | − | − |
| P213 | V | 12 | A + (B) | − | − | − | − | ± | − | − | − | − | − | P* |
| P214 | V | 12 | A + (B) | − | − | − | − | − | − | − | − | − | − | − |
| | | | # of positive | 0/24 | 0/24 | 0/24 | 3/24 | 4/24 | 5/24 | 4/24 | 3/24 | 3/24 | 4/24 | 10/24 (5*/24) |
| | | | % positive | 0% | 0% | 0% | 12.5% | 16.7% | 20.8% | 16.7% | 12.5% | 12.5% | 16.7% | 41.7 (20.8*)% |
| G197 | C | 12 | A + B | − | − | − | ++ | +++ | +++ | ++++ | +++ | ++ | ++ | P |
| G205 | C | 12 | A + B | − | − | − | ++ | +++ | +++ | ++++ | +++ | + | + | P |
| G293 | C | 11 | A | − | − | − | − | − | ++ | + | ± | ± | − | P |
| G296 | C | 11 | A | − | − | − | − | − | ++ | ++++ | + | + | + | P |
| O109 | C | 11-13 | A | − | − | − | − | − | ++ | ++++ | +++ | ++ | +++ | P |
| O110 | C | 11-13 | A | − | − | − | − | − | + | +++ | + | + | + | P |
| O111 | C | 11-13 | A | − | − | − | − | − | ++ | ++ | ++ | + | + | P |
| O114 | C | 11-13 | A | − | − | − | − | − | + | − | + | − | − | P |
| O162 | C | 12 | A + B | − | − | − | + | +++ | ++ | ++++ | +++ | ++ | NA | P |
| O164 | C | 12 | A | − | − | − | − | ± | + | +++ | ++ | + | ± | P |
| O166 | C | 12 | A + B | − | − | − | − | ++ | ++ | +++ | ++ | ++ | + | P |
| P105 | C | 12-13 | A + B | − | − | − | − | ++++ | ++ | ++++ | ++ | ++ | + | P |
| P107 | C | 12-13 | A + B | − | − | − | + | ++++ | ++ | +++ | ++ | + | + | P |
| P108 | C | 12-13 | A + B | − | − | ++++ | ++++ | ++++ | +++ | ++++ | +++ | +++ | NA | P |
| P110 | C | 12-13 | A + B | − | − | − | ++ | +++ | ++ | +++ | + | + | +++ | P |
| P112 | C | 11 | A | − | − | − | − | − | ++ | ++ | + | − | + | P |
| P116 | C | 11 | A | − | − | − | − | − | + | ++ | + | + | + | P |
| P147 | C | 11 | A | − | − | − | − | − | − | − | ± | − | − | P* |
| P148 | C | 11 | A | − | − | − | − | − | + | + | + | +++ | + | P |
| P194 | C | 11 | A | − | − | − | − | − | ± | + | + | ± | + | P |
| P195 | C | 11 | A | − | − | − | − | − | + | ++ | + | + | +++ | P |
| P215 | C | 12 | A + B | − | − | − | − | + | + | ++ | + | + | + | P |
| P216 | C | 12 | A | − | − | − | − | − | + | + | ± | ++ | + | P |
| P218 | C | 12 | A + B | − | − | − | − | + | + | + | + | ± | − | P |
| | | | # of positive | 0/24 | 0/24 | 1/24 | 6/24 | 11/24 | 23/24 | 22/24 | 24/24 | 21/24 | 18/22 | 24/24 (23*/24) |
| | | | % positive | 0% | 0% | 4.2% | 25.0% | 45.8% | 95.8% | 91.7% | 100% | 87.5% | 81.8% | 100 (95.8*)% |

V = Vaccinate Group;
C = Control Group;
NA = not determined;
Rooms 11 or 12: piglets were housed during whole study (vaccination and challenge phases);
Rooms 11-13 and 12-13: piglets were housed in Room 11 or 12 during vaccination phase and relocated into Room 13 on 0DPC (challenge phase).
PCV2 Challenge Strain: A = experimental challenge-PCV2 #40895; A + (B) = In addition to experimental challenge-PCV2 #40895, potential environmental contact challenge due to intermingling with Pig #108 during either vaccination or challenge phase. However, these pigs were protected from PCV2 challenge, and no PCR product can be sequenced for PCV2B; A + B = experimental challenge-PCV2 #40895, environmental contact challenge due to Pig #108, and PCR product was sequenced and identified as PCV2B from the same source (Pig #108)
Score of PCR band intensity: − Negative; ± Very faint marginally visible PCR band; + Positive; ++ Strong positive; +++ Very strong positive; ++++ Extremely strong positive;
PCV2 viremia: − = Negative; P = positive; P* = inconclusive (a single occurrence or two of inconclusive PCR band)

TABLE 3B
Detection of PCV2 Viremia by PCV2-Specific PCR in Sera of Vaccinated and Control Pigs: Experimental PCV2
(#40895) Challenge

| | | | PCV2 | 0 | 13 | 28 | −1 | 3 | 7 | 10 | 14 | 17 | 20 | PCV2 |
|---|---|---|---|---|---|---|---|---|---|---|---|---|---|---|
| PIG ID | GROUP | ROOM | CHALLENGE | DPV | DPV | DPV | DPC | DPC | DPC | DPC | DPC | DPC | DPC | Viremia |
| G289 | V | 11 | A | − | − | − | − | − | ± | − | − | − | − | P* |
| G290 | V | 11 | A | − | − | − | − | − | ± | − | − | − | − | P* |
| O119 | V | 11-13 | A | − | − | − | − | − | − | + | − | − | − | P |
| P113 | V | 11 | A | − | − | − | − | − | − | − | − | − | − | − |
| P115 | V | 11 | A | − | − | − | − | − | − | − | − | − | − | − |
| P145 | V | 11 | A | − | − | − | − | − | − | − | − | − | − | − |
| P146 | V | 11 | A | − | − | − | − | − | ± | − | − | − | ± | P* |
| P150 | V | 11 | A | − | − | − | − | − | − | − | − | − | − | − |
| P192 | V | 11 | A | − | − | − | − | − | − | − | − | − | − | − |
| | | | # of positive | 0/9 | 0/9 | 0/9 | 0/9 | 0/9 | 2/9 | 1/9 | 0/9 | 0/9 | 1/9 | 4/9 (1*/9) |
| | | | % positive | 0% | 0% | 0% | 0% | 0% | 22.2% | 11.1% | 0% | 0% | 11.1% | 44.4 |
| | | | | | | | | | | | | | | (11.1*)% |
| G293 | C | 11 | A | − | − | − | − | − | ++ | + | ± | ± | − | P |
| G296 | C | 11 | A | − | − | − | − | − | ++ | ++++ | + | + | + | P |
| O109 | C | 11-13 | A | − | − | − | − | − | ++ | ++++ | +++ | ++ | +++ | P |
| O110 | C | 11-13 | A | − | − | − | − | − | + | +++ | + | + | + | P |
| O111 | C | 11-13 | A | − | − | − | − | − | ++ | ++ | ++ | + | + | P |
| O114 | C | 11-13 | A | − | − | − | − | − | + | − | + | − | − | P |
| O164 | C | 12 | A | − | − | − | − | ± | + | +++ | ++ | + | ± | P |
| P112 | C | 11 | A | − | − | − | − | − | ++ | ++ | + | − | + | P |
| P116 | C | 11 | A | − | − | − | − | − | + | ++ | + | + | + | P |
| P147 | C | 11 | A | − | − | − | − | − | − | − | ± | − | − | P* |
| P148 | C | 11 | A | − | − | − | − | − | + | + | + | +++ | + | P |
| P194 | C | 11 | A | − | − | − | − | − | ± | + | + | ± | + | P |
| P195 | C | 11 | A | − | − | − | − | − | + | ++ | + | + | +++ | P |
| P216 | C | 12 | A | − | − | − | − | − | + | + | ± | ++ | + | p |
| | | | # of positive | 0/14 | 0/14 | 0/14 | 0/14 | 1/14 | 13/14 | 12/14 | 14/14 | 11/14 | 11/14 | 14/14 |
| | | | | | | | | | | | | | | (13*/14) |
| | | | % positive | 0% | 0% | 0% | 0% | 7.1% | 92.9% | 85.7% | 100% | 78.6% | 78.6% | 100 |
| | | | | | | | | | | | | | | (92.9*)% |

V = Vaccinate Group;
C = Control Group;
NA = not determined
Rooms 11 and 12: piglets were housed during whole study (vaccination and challenge phases);
Rooms 11-13 and 12-13: piglets were housed in Room 11 or 12 during vaccination phase and relocated into Room 13 on 0DPC (challenge phase).
PCV2 Challenge Strain: A = experimental challenge-PCV2 #40895
Score of PCR band intensity: − Negative; ± Very faint marginally visible PCR band; + Positive; ++ Strong positive; +++ Very strong positive; ++++ Extremely strong positive
PCV2 viremia: − = Negative; P = positive; P* = inconclusive (a single occurrence or two of inconclusive PCR band)

TABLE 3C
Detection of PCV2 Viremia by PCV2-Specific PCR in Sera of Vaccinated and Control Pigs: Experimental PCV2
(#40895) and Environmental Contact Challenges

| | | | PCV2 | 0 | 13 | 28 | −1 | 3 | 7 | 10 | 14 | 17 | 20 | PCV2 |
|---|---|---|---|---|---|---|---|---|---|---|---|---|---|---|
| PIG ID | GROUP | ROOM | CHALLENGE | DPV | DPV | DPV | DPC | DPC | DPC | DPC | DPC | DPC | DPC | Viremia |
| G199 | V | 12 | A + (B) | − | − | − | − | − | − | − | − | − | − | − |
| G203 | V | 12 | A + B | − | − | − | − | + | ++ | ++++ | +++ | ++ | ++ | P |
| G204 | V | 12 | A + B | − | − | − | − | ++ | ++ | ++ | ++ | + | + | P |
| O112 | V | 11-13 | A + (B) | − | − | − | − | − | − | − | − | − | − | − |
| O113 | V | 11-13 | A + (B) | − | − | − | − | − | − | − | − | − | − | − |
| O117 | V | 11-13 | A + (B) | − | − | − | − | − | − | − | − | − | − | − |
| O156 | V | 12 | A + (B) | − | − | − | − | − | − | − | − | − | − | − |
| O158 | V | 12 | A + (B) | − | − | − | − | − | − | − | − | − | − | − |
| O159 | V | 12 | A + (B) | − | − | − | − | − | − | − | − | − | − | − |
| P102 | V | 12-13 | A + B | − | − | − | + | − | − | − | − | − | − | P |
| P103 | V | 12-13 | A + B | − | − | − | ± | − | − | − | − | − | − | P* |
| P104 | V | 12-13 | A + B | − | − | − | ++ | ++++ | ++ | +++ | +++ | + | ± | P |
| P109 | V | 12-13 | A + (B) | − | − | − | − | − | − | − | − | − | − | − |
| P213 | V | 12 | A + (B) | − | − | − | − | ± | − | − | − | − | − | P* |
| P214 | V | 12 | A + (B) | − | − | − | − | − | − | − | − | − | − | − |
| | | | # of positive | 0/15 | 0/15 | 0/15 | 3/15 | 4/15 | 3/15 | 3/15 | 3/15 | 3/15 | 3/15 | 6/15 (4*/15) |
| | | | % positive | 0% | 0% | 0% | 20% | 26.7% | 20% | 20% | 20% | 20% | 20% | 40 (26.7*)% |
| G197 | C | 12 | A + B | − | − | − | ++ | +++ | +++ | ++++ | +++ | ++ | ++ | P |
| G205 | C | 12 | A + B | − | − | − | ++ | +++ | +++ | ++++ | +++ | + | + | P |
| O162 | C | 12 | A + B | − | − | − | + | +++ | ++ | ++++ | +++ | ++ | NA | P |
| O166 | C | 12 | A + B | − | − | − | − | ++ | ++ | +++ | ++ | ++ | + | P |
| P105 | C | 12-13 | A + B | − | − | − | − | ++++ | ++ | ++++ | ++ | ++ | + | p |
| P107 | C | 12-13 | A + B | − | − | − | + | ++++ | ++ | +++ | ++ | + | + | P |
| P108 | C | 12-13 | A + B | − | − | ++++ | ++++ | ++++ | +++ | ++++ | +++ | +++ | NA | P |
| P110 | C | 12-13 | A + B | − | − | − | ++ | +++ | ++ | +++ | + | + | +++ | P |
| P215 | C | 12 | A + B | − | − | − | − | + | + | ++ | + | + | + | P |
| P218 | C | 12 | A + B | − | − | − | − | + | + | + | + | ± | − | P |
| | | | # of positive | 0/10 | 0/10 | 1/10 | 6/10 | 10/10 | 10/10 | 10/10 | 10/10 | 10/10 | 7/8 | 10/10 |
| | | | % positive | 0% | 0% | 10% | 60% | 100% | 100% | 100% | 100% | 100% | 87.5% | 100% |

V = Vaccinate Group;
C = Control Group;
NA = not determined
Rooms 11 and 12: piglets were housed during whole study (vaccination and challenge phases);
Rooms 11-13 and 12-13: piglets were housed in Room 11 or 12 during vaccination phase and relocated into Room 13 on 0DPC (challenge phase).
PCV2 Challenge Strain: A + (B) = In addition to experimental challenge-PCV2 #40895, potential environmental contact challenge due to intermingling with Pig #108 during either vaccination or challenge phase. However, these pigs were protected from PCV2 challenge, and no PCR product can be sequenced for PCV2B; A + B = experimental challenge-PCV2 #40895, environmental contact challenge due to Pig #108, and PCR product was sequenced and identified as PCV2B from the same source (Pig #108)
Score of PCR band intensity: − Negative; ± Very faint marginally visible PCR band; + Positive; ++ Strong positive; +++ Very strong positive; ++++ Extremely strong positive
PCV2 viremia: − = Negative; P = positive; P* = inconclusive (a single occurrence or two of inconclusive PCR band)

TABLE 4A
Lymphoid Depletion in Lymph Nodes,
Spleen and Tonsil: Overall Comparison*

Number of
Animals
with Score*

| | | | | |
|---|---|---|---|---|
| Group | 0 | 1 | 2 | 3 |

Iliac Lymph Node (IL LN)

| | | | | |
|---|---|---|---|---|
| Vaccinate | 13 | 7 | 2 | 2 |
| Control | 4 | 9 | 3 | 8 |

Inguinal Lymph Node (IG LN)

| | | | | |
|---|---|---|---|---|
| Vaccinate | 15 | 7 | 1 | 1 |
| Control | 5 | 10 | 6 | 3 |

Tracheobronchial Lymph Node (TB LN)

| | | | | |
|---|---|---|---|---|
| Vaccinate | 12 | 11 | 0 | 1 |
| Control | 2 | 9 | 9 | 4 |

Spleen

| | | | | |
|---|---|---|---|---|
| Vaccinate | 15 | 9 | 0 | 0 |
| Control | 7 | 6 | 8 | 3 |

Tonsil

| | | | | |
|---|---|---|---|---|
| Vaccinate | 19 | 4 | 1 | 0 |
| Control | 10 | 4 | 7 | 3 |

*Lymphoid depletion score: 0 = Normal, 1 = Mild, 2 = Moderate, and 3 = Severe

TABLE 4B
Lymphoid Depletion in Lymph Nodes, Spleen and Tonsil:
Experimental PCV2 (#40895) Challenge

Number of
Animals
with Score*

| | | | | |
|---|---|---|---|---|
| Group | 0 | 1 | 2 | 3 |

Iliac Lymph Node (IL LN)

| | | | | |
|---|---|---|---|---|
| Vaccinate | 4 | 4 | 0 | 1 |
| Control | 3 | 7 | 2 | 2 |

Inguinal Lymph Node (IG LN)

| | | | | |
|---|---|---|---|---|
| Vaccinate | 6 | 3 | 0 | 0 |
| Control | 5 | 7 | 2 | 0 |

Tracheobronchial Lymph Node (TB LN)

| | | | | |
|---|---|---|---|---|
| Vaccinate | 4 | 5 | 0 | 0 |
| Control | 2 | 8 | 3 | 1 |

Spleen

| | | | | |
|---|---|---|---|---|
| Vaccinate | 6 | 3 | 0 | 0 |
| Control | 7 | 5 | 2 | 0 |

Tonsil

| | | | | |
|---|---|---|---|---|
| Vaccinate | 9 | 0 | 0 | 0 |
| Control | 10 | 2 | 2 | 0 |

*Lymphoid depletion score: 0 = Normal, 1 = Mild, 2 = Moderate, and 3 = Severe

TABLE 4C
Lymphoid Depletion in Lymph Nodes, Spleen and Tonsil:
Experimental PCV2A (#40895) and Environmental Contact
Challenges

Number of
Animals
with Score*

| | | | | |
|---|---|---|---|---|
| Group | 0 | 1 | 2 | 3 |

Iliac Lymph Node (IL LN)

| | | | | |
|---|---|---|---|---|
| Vaccinate | 9 | 3 | 2 | 1 |
| Control | 1 | 2 | 1 | 6 |

Inguinal Lymph Node (IG LN)

| | | | | |
|---|---|---|---|---|
| Vaccinate | 9 | 4 | 1 | 1 |
| Control | 0 | 3 | 4 | 3 |

Tracheobronchial Lymph Node (TB LN)

| | | | | |
|---|---|---|---|---|
| Vaccinate | 8 | 6 | 0 | 1 |
| Control | 0 | 1 | 6 | 3 |

Spleen

| | | | | |
|---|---|---|---|---|
| Vaccinate | 9 | 6 | 0 | 0 |
| Control | 0 | 1 | 6 | 3 |

Tonsil

| | | | | |
|---|---|---|---|---|
| Vaccinate | 10 | 4 | 1 | 0 |
| Control | 0 | 2 | 5 | 3 |

*Lymphoid depletion score: 0 = Normal, 1 = Mild, 2 = Moderate, and 3 = Severe

TABLE 5
Histiocytic-to-Granulomatous Inflammation with Replacement
of Follicles in Lymph Nodes, Spleen and Tonsil: Overall
Comparison

Number of
Animals
with Score*

| | | | | |
|---|---|---|---|---|
| Group | 0 | 1 | 2 | 3 |

Iliac Lymph Node (IL LN)

| | | | | |
|---|---|---|---|---|
| Vaccinate | 14 | 2 | 7 | 1 |
| Control | 4 | 8 | 7 | 5 |

Inguinal Lymph Node (IG LN)

| | | | | |
|---|---|---|---|---|
| Vaccinate | 18 | 3 | 3 | 0 |
| Control | 8 | 9 | 4 | 3 |

Tracheobronchial Lymph Node (TB LN)

| | | | | |
|---|---|---|---|---|
| Vaccinate | 14 | 7 | 3 | 0 |
| Control | 4 | 9 | 9 | 2 |

Spleen

| | | | | |
|---|---|---|---|---|
| Vaccinate | 18 | 2 | 4 | 0 |
| Control | 9 | 4 | 8 | 3 |

Tonsil

| | | | | |
|---|---|---|---|---|
| Vaccinate | 19 | 4 | 1 | 0 |
| Control | 11 | 5 | 4 | 4 |

*Histiocytic replacement score: 0 = Normal, 1 = Mild, 2 = Moderate, and 3 = Severe

TABLE 6
Amount of PCV2-Specific Antigen Demonstrated by
Immunohistochemistry (IHC) in Lymph Nodes, Spleen
and Tonsil: Overall Comparison*

Number of
Animals
with Score*

| | | | | |
|---|---|---|---|---|
| Group | 0 | 1 | 2 | 3 |

Iliac Lymph Node (IL LN)

| | | | | |
|---|---|---|---|---|
| Vaccinate | 17 | 2 | 5 | 0 |
| Control | 4 | 6 | 7 | 7 |

Inguinal Lymph Node (IG LN)

| | | | | |
|---|---|---|---|---|
| Vaccinate | 21 | 1 | 2 | 0 |
| Control | 10 | 9 | 4 | 1 |

Tracheobronchial Lymph Node (TB LN)

| | | | | |
|---|---|---|---|---|
| Vaccinate | 20 | 2 | 1 | 1 |
| Control | 7 | 12 | 3 | 2 |

Spleen

| | | | | |
|---|---|---|---|---|
| Vaccinate | 20 | 2 | 2 | 0 |
| Control | 9 | 9 | 4 | 2 |

Tonsil

| | | | | |
|---|---|---|---|---|
| Vaccinate | 21 | 1 | 2 | 0 |
| Control | 5 | 10 | 6 | 3 |

IHC staining score: 0 = No, 1 = Low Level, 2 = Medium Level, and 3 = High Level

TABLE 7
Post-Challenge Clinical Observations*

| PIG | | | | | | | | | | | | | | | |
|---|---|---|---|---|---|---|---|---|---|---|---|---|---|---|---|
| ID | GROUP | −2DPC | −1DPC | 0 DPC | 1 DPC | 2 DPC | 3 DPC | 4 DPC | 5 DPC | 6 DPC | 7 DPC | 10 DPC | 14 DPC | 17 DPC | 20 DPC |
| G199 | V | A | A | A | A | A | A | A | A | A | A | A | A | A | A |
| G203 | V | A | A | A | A | A | A | A | A | A | A | A | A | A | A |
| G204 | V | A | A | A | A | A | A | A | A | A | A | A | A | A | A |
| G289 | V | A | A | A | A | A | A | A | A | A | A | A | A | A | A |
| G290 | V | A | A | A | A | A | A | A | A | A | A | A | A | A | A |
| O112 | V | A | A | A | A | A | A | A | A | A | A | A | A | A | A |
| O113 | V | A | A | A | A | A | A | A | A | A | A | A | A | A | A |
| O117 | V | A | A | A | A | A | A | A | A | A | A | A | A | A | A |
| O119 | V | A | A | A | A | A | A | A | A | A | A | A | A | A | A |
| O156 | V | A | A | A | A | A | A | A | A | A | A | A | A | A | A |
| O158 | V | A | A | A | A | A | A | A | A | A | A | A | A | A | A |
| O159 | V | A | A | A | A | A | A | A | A | A | A | A | A | A | A |
| P102 | V | A | A | A | A | A | A | A | A | A | A | A | A | A | A |
| P103 | V | A | A | A | A | A | A | A | A | A | A | A | A | A | A |
| P104 | V | A | A | A | A | A | A | A | A | A | A | A | A | A | A |
| P109 | V | A | A | A | A | A | A | A | A | A | A | A | A | A | A |
| P113 | V | A | A | A | A | A | A | A | A | A | A | A | A | A | A |
| P115 | V | A | A | A | A | A | A | A | A | A | A | A | A | A | A |
| P145 | V | A | A | A | A | A | A | A | A | A | A | A | A | A | A |
| P146 | V | A | A | A | A | A | A | A | A | A | A | A | A | A | A |
| P150 | V | A | A | A | A | A | A | A | A | A | A | A | A | A | A |
| P192 | V | A | A | A | A | A | A | A | A | A | A | A | A | A | A |
| P213 | V | A | A | A | A | A | A | A | A | A | A | A | A | A | A |
| P214 | V | A | A | A | A | A | A | A | A | A | A | A | A | A | A |
| G197 | C | A | A | A | A | A | A | A | A | A | A | A | A | A | A |
| G205 | C | A | A | A | A | A | A | A | A | A | A | A | A | A | A |
| G293 | C | A | A | A | A | A | A | A | A | A | A | A | A | A | A |
| G296 | C | A | A | A | A | A | A | A | A | A | A | A | A | A | A |
| O109 | C | A | A | A | A | A | A | A | A | A | A | A | A | A | A |
| O110 | C | A | A | A | A | A | A | A | A | A | A | A | A | A | A |
| O111 | C | A | A | A | A | A | A | A | A | A | A | A | A | A | A |
| O114 | C | A | A | A | A | A | A | A | A | A | A | A | A | A | A |
| O162 | C | A | A | A | A | A | A | A | A | A | A | A | I | I | . |
| O164 | C | A | A | A | A | A | A | A | A | A | A | A | A | A | A |
| O166 | C | A | A | A | A | A | A | A | A | A | A | A | A | A | A |
| P105 | C | A | A | A | A | A | A | A | A | A | A | A | A | A | A |
| P107 | C | A | A | A | A | A | A | A | A | A | A | A | A | A | A |
| P108 | C | A | A | A | E | A | A | A | A | A | A | E, I | I | I | . |
| P110 | C | A | A | A | A | A | A | A | A | A | A | A | A | A | A |
| P112 | C | A | A | A | A | A | A | A | A | A | A | A | A | A | A |
| P116 | C | A | A | A | A | A | A | A | A | A | A | A | A | A | A |
| P147 | C | A | A | A | A | A | A | A | A | A | A | A | A | A | A |
| P148 | C | A | A | A | A | A | A | A | A | A | A | A | A | A | A |
| P194 | C | A | A | A | A | A | A | A | A | A | A | A | A | A | A |
| P195 | C | A | A | A | A | A | A | A | A | A | A | A | A | A | A |
| P215 | C | A | A | A | A | A | A | A | A | A | A | A | A | A | A |
| P216 | C | A | A | A | A | A | A | A | A | A | A | A | A | A | A |
| P218 | C | A | A | A | A | A | A | A | A | A | A | A | A | A | A |

*A = Normal; E = Coughing; I = Others (see comments in Section 6.8)
V = Vaccinate Group;
C = Control Group
. = NA

### SUMMARY

Two new strains of type 2B pathogenic porcine circoviruses have been isolated from pigs in the present study. The identification and isolation of these two new strains of type 2B circovirus from pig numbers P108 and O162 in the study described herein will allow for the preparation of immunogenic or vaccine compositions for protection of pigs against PMWS.

What is claimed is:

1. An isolated porcine circovirus whose genome comprises the nucleic acid molecule of either of SEQ ID NOs: 1 or 2, or whose genome comprises a nucleic acid molecule having at least 95% sequence homology to either of SEQ ID NOs: 1 or 2.

2. The isolated porcine circovirus of claim 1, whose genome comprises the nucleic acid molecule of either of SEQ ID NOs: 1 or 2.

3. The isolated porcine circovirus of claim 1, which is a type 2B porcine circovirus.

4. The isolated porcine circovirus of claim 3, having an ORF2 protein with at least 92% sequence identity to either of SEQ ID NOs: 3 or 4.

5. The isolated porcine circovirus of claim 3, wherein the nucleic acid encoding the ORF2 protein comprises residues 1033-1734 of SEQ ID NOs: 5 or 6.

6. An isolated nucleic acid molecule encoding a pathogenic type 2B porcine circovirus, or encoding at least one protein from said circovirus, wherein the nucleic acid molecule comprises the nucleotide sequence of any of SEQ ID NOs: 1, 2, 5 or 6 or a nucleotide sequence having at least 95% sequence homology to any of SEQ ID NOs:1, 2, 5 or 6.

7. The isolated nucleic acid molecule of claim 6, comprising the nucleotide sequence of any of SEQ ID NOs: 1, 2, 5 or 6.

8. The isolated nucleic acid molecule of claim 6, wherein the nucleic acid encoding an ORF2 protein is found at residues 1033-1734 of SEQ ID NOs: 5 or 6.

9. The isolated nucleic acid molecule of claim 8, which encodes an ORF2 protein having the amino acid sequence as set forth in SEQ ID NO: 3 or 4.

10. An immunogenic composition comprising the isolated porcine circovirus of claim 1, and a pharmaceutically acceptable adjuvant.

11. The immunogenic composition of claim 10, wherein the isolated porcine circovirus is attenuated or inactivated.

12. The immunogenic composition of claim 11, further comprising at least one other microorganism, or an antigen obtained from said microorganism against which an immune response is desired.

13. The immunogenic composition of claim 12, wherein the other microorganism is selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, a pathogen causative of Swine Transmissible Gastroenteritis, and a second different strain of porcine circovirus.

14. The immunogenic composition of claim 13, wherein the second different strain of porcine circovirus is a type 2A or a type 2B circovirus.

15. An immunogenic composition comprising at least one isolated nucleic acid molecule of claim 6, and a pharmaceutically acceptable adjuvant.

16. The immunogenic composition of claim 15, further comprising a nucleic acid molecule encoding at least one antigen from at least one other microorganism against which an immune response is desired.

17. The immunogenic composition of claim 16, wherein the other microorganism is selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, a pathogen causative of Swine Transmissible Gastroenteritis and a second different strain of porcine circovirus.

18. The immunogenic composition of claim 17, wherein the second different strain of porcine circovirus is a type 2A or a type 2B circovirus.

19. The composition of claim 10, wherein the composition is administered in one dose or in multiple doses subcutaneously, intramuscularly, intranasally, transdermally, intrahepatically, or via the intralymphoid route.

20. A method of immunizing a pig against viral infection or postweaning multisystemic wasting syndrome (PMWS), or for preventing postweaning multisystemic wasting syndrome (PMWS) in a pig caused by a strain of PCV2, comprising administering to the pig an immunogenically effective amount of a composition comprising any one or more of the following:
a) an immunogenically effective amount of the type 2 porcine circovirus of claim 1;
b) a nucleic acid molecule encoding the type 2 porcine circovirus of a);
c) an immunogenically effective amount of at least one protein isolated from the type 2 porcine circovirus of claim 1; or
d) a nucleic acid molecule encoding at least one protein of c).

21. The method of claim 20, wherein the method further comprises administering an immunogenically effective amount of a second different immunogenic composition prior to, in conjunction with, or subsequent to, administering the type 2 porcine circovirus immunogenic composition.

22. The method of claim 21, wherein the second different immunogenic composition comprises an immunogenically effective amount of at least one other microorganism that is pathogenic to pigs, or at least one antigen obtained from said microorganism or a nucleic acid molecule encoding said antigen, wherein the microorganism is selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, a pathogen causative of Swine Transmissible Gastroenteritis and a second different strain of porcine circovirus.

23. The method of claim 22, wherein the second different strain of porcine circovirus is a type 2A or a 2B circovirus.

24. A vector comprising at least one exogenous nucleic acid molecule encoding a type 2A or type 2B porcine circovirus protein, wherein the porcine circovirus protein is an ORF2 protein, and wherein the exogenous nucleic acid molecule encoding said protein is set forth in residues 1033-1734 of SEQ ID NOs: 5 or 6.

25. The vector of claim 24, wherein the vector is a raccoon poxvirus vector.

26. The vector of claim 24, further comprising one or more exogenous nucleic acid molecules encoding an antigen from a microorganism that is pathogenic to pigs, wherein the microorganism is selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, a pathogen causative of Swine Transmissible Gastroenteritis and a second different strain of porcine circovirus.

27. A method of determining if a porcine mammal has, or is at risk for developing postweaning multisystemic wasting syndrome (PMWS), the method comprising:
(I) measuring an amount of a PCV2 nucleic acid or protein encoded by said nucleic acid in a tissue sample derived from the mammal, wherein said PCV2 nucleic acid or protein is:
a) a nucleic acid corresponding to any of SEQ ID NOs: 1, 2, 5 or 6, or a nucleic acid derived therefrom;
b) a protein comprising either of SEQ ID NOs: 3 or 4;
c) a nucleic acid comprising a sequence hybridizable to any of SEQ ID NOs: 1 , 2, 5 or 6, or their complements under conditions of high stringency, or a protein comprising a sequence encoded by said hybridizable sequence;
d) a nucleic acid at least 95% homologous to any of SEQ ID NOs: 1, 2, 5, or 6. or their complement as determined using the NBLAST algorithm; or a protein encoded thereby; and
(II) comparing the amount of said nucleic acid or protein in the tissue sample from the mammal suspected of having, or at risk for developing PMWS with the amount of nucleic acid or protein present in a tissue sample from a normal mammal, or predetermined standard for a normal tissue sample, wherein an elevated amount of said nucleic acid or protein in the tissue sample from the porcine mammal having or suspected of having PMWS compared to the amount in the normal tissue sample or pre-determined standard for a normal tissue sample indicates that the mammal has or is at risk of developing PMWS.

28. The method of claim 27, wherein the tissue sample is selected from the group consisting of inguinal superficial lymph node, tracheobronchial lymph node, submandibular lymph node, lung, tonsil, spleen, liver, kidney, whole blood and blood cells.


SEQUENCE LISTING (8 sequences)

SEQ ID NO: 1 - DNA, 1767 bp, Porcine circovirus
accagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagccaaaggaacagatcagcagaataaagaatactgcagtaaagaaggcaacttactgattgagtgtggagctcctagatctcagggacaacggagtgacctgtctactgctgtgagtaccttgttggagagcgggagtctggtgaccgttgcagagcagtaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaacgcagtgccgaggcctacgtggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgatagtatatccgaaggtgcgggataggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatagctgaaaacgaaagaagtgcgctgtaagtatt

SEQ ID NO: 2 - DNA, 1767 bp, Porcine circovirus
accagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagccaaaggaacagatcagcagaataaagaatactgcagtaaagaaggtaacttactgattgagtgtggagctcctagatctcagggacaacggagtgacctgtctactgctgtgagtaccttgttggagagcgggagtctggtgaccgttgcagagcagtaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaacgcagtgccgaggcctacgtggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgatagtgtatccgaaggtgcgggataggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 3 - protein, 233 aa, Porcine circovirus
MTYPRRRYRRRRHRPRSHLGQILRRRPWLVHPRHRYRWRRKNGIFNTRLSRTFGYTIKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNLKDPPLNP

SEQ ID NO: 4 - protein, 233 aa, Porcine circovirus; identical to SEQ ID NO: 3

SEQ ID NO: 5 - DNA, 1767 bp, Porcine circovirus; identical to SEQ ID NO: 1
  gene (51)..(995): replicase gene (ORF1)

SEQ ID NO: 6 - DNA, 1767 bp, Porcine circovirus; identical to SEQ ID NO: 2
  gene (51)..(995): replicase gene (ORF1)

SEQ ID NO: 7 - DNA, 20 bp, Artificial sequence
  primer
atgcccagcaagaagaatgg

SEQ ID NO: 8 - DNA, 22 bp, Artificial sequence
  primer
tggtttccagtatgtggtttcc